(19) United States
(12) Patent Application Publication
(10) Pub. No.: US 2009/0136460 A1
(43) Pub. Date: May 28, 2009

(54) USE OF ADENOSINE RECEPTOR LIGANDS TO PROMOTE CELL ADHESION IN CELL-BASED THERAPIES

(75) Inventors: Italo Biaggioni, Nashville, TN (US); Igor Feoktistov, Nashville, TN (US); Antonis K. Hatzopoulos, Nashville, TN (US); Sergey Ryzhov, Nashville, TN (US); Douglas E. Vaughan, Chicago, IL (US)

Correspondence Address: FULBRIGHT & JAWORSKI L.L.P., 600 CONGRESS AVE., SUITE 2400, AUSTIN, TX, 78701, US

(73) Assignee: VANDERBILT UNIVERSITY

(21) Appl. No.: 12/170,883

(22) Filed: Jul. 10, 2008

Related U.S. Application Data
(60) Provisional application No. 60/948,886, filed on Jul. 10, 2007.

(51) Int. Cl. A61K 35/12

(52) U.S. Cl. 424/93.7

(57) ABSTRACT

Intracoronary delivery of endothelial progenitor cells (EPCs) is an emerging concept for the treatment of cardiovascular disease, and enhancement of EPC adhesion to vascular endothelium should improve cell retention within targeted organs, as well as vascular development. The present inventors have shown that stimulation of adenosine receptors (AdoR) in murine embryonic EPCs (eEPCs) and cardiac endothelial cells (cECs) rapidly, within minutes, increased eEPC adhesion to cECs. eEPCs and cECs were found to predominantly express functional A₁ and A_{2B} AdoR subtypes, respectively, and both subtypes are involved in the regulation of eEPC adhesion to cECs. Adenosine, adenosine precursors (e.g., AMP) and adenosine receptor agonists thus can be used to stimulate EPC/stem cell homing and engraftment in cell-based therapies.

The present application claims benefit of priority to U.S. Provisional Application Ser. No. 60/948,886, filed Jul. 10, 2007, the entire contents of which are hereby incorporated by reference.

This invention was made with government support under grant nos. R01 HL76306 and R01 HL083958 awarded by the National Institutes of Health. The government has certain rights in the invention.

### BACKGROUND OF THE INVENTION

1. Field of the Invention

The present invention relates generally to the fields of pathology and cardiology. More particularly, it concerns the use of adenosine, adenosine precursors (e.g., AMP), adenosine potentiators (e.g., dipyridamole) and adenosine receptor agonists to promote adhesion of stem cells (e.g., endothelial progenitor cells) to cardiac endothelium.

2. Description of Related Art

Heart Failure (HF) is the most common reason for admission to hospitals in the United States, especially in the Medicare population. There are approximately 1 million new cases diagnosed each year and this number will grow as the population ages. A majority of patients with HF have poor left ventricular function, with 60-70% due to chronic ischemic coronary disease. The remainder of patients experience HF due to a variety of other causes, including idiopathic and viral cardiomyopathies, and are classified as dilated non-ischemic cardiomyopathies.

Until several years ago, it was felt that the cardiac damage was irreversible and treatment alternatives for these patients were limited to medical therapy to preserve residual heart function, revascularization to prevent further myocyte death, or heart transplantation. Recently, there have been a number of reports indicating that stem cells (endothelial, mesenchymal, or skeletal) or stem-cell enriched preparations from bone marrow, injected directly into the myocardium or delivered to the coronary circulation, can improve cardiac function in chronic ischemic cardiomyopathy or following an acute myocardial infarction. More recently, studies employing a variety of cellular preparations and delivery strategies have been extended into clinical populations.

A similar approach can be used to treat patients with coronary artery disease, by promoting revascularization.

Intracoronary injection of bone marrow-derived stem cells or culture-expanded endothelial progenitor cells is currently tested for the treatment of patients after acute myocardial infarction. Recent double-blinded, placebo-controlled, multi-center clinical trials have shown that this type of therapy is relatively safe without serious adverse effects and may lead to moderate improvement of cardiac output (Schachinger et al., 2006; Bartunek et al., 2007). However, the number of donor cells retained in the heart is low, in the range of 3-5% (Aicher et al., 2003), limiting the effectiveness of therapy. To overcome this problem, it would be highly desirable to develop methods to improve adhesion and retention of endothelial progenitor cells to cardiac endothelium.

The inventors have previously shown that homing of endothelial progenitor cells (EPCs) to sites of tumor-induced angiogenesis or cardiac ischemia is mediated by active interaction with the vascular wall (Vajkoczy et al., 2003; Kupatt et al., 2005) suggesting that pre-activation of adhesive molecules in host endothelium and donor transplanted cells might augment cell retention in target tissues. However, activation of cell adhesion molecules in endothelial cells after ischemic injury or inflammation is likely to be transient and absent by the time of therapeutic intervention. Therefore, there is a need to develop safe ways to activate, locally and acutely, the adhesiveness of vascular beds during cell delivery.

Adenosine may represent an ideal adjunct agent to cell-based therapy in treatment of cardiovascular disease. This nucleoside is generated when ATP is catabolized as energy demands increase or oxygen supply decreases in sites of tissue stress, injury and local hypoxia. Adenosine exerts its actions through interaction with cell surface G protein-coupled adenosine receptors, of which there are four subtypes, A₁, A_{2A}, A_{2B} and A₃ (Fredholm et al., 2001). Once released into the extracellular space, adenosine signals to restore the balance between energy supply and demand. Originally proposed by Berne et al, this concept of adenosine as a retaliatory autacoid has focused mostly on its acute actions, including vasodilation and negative chronotropic and inotropic effects in the heart (Berne et al., 1983).

Accumulating evidence suggests that adenosine is also important for the long-term restoration of oxygen supply by contributing to neovascularization. For example, it was shown that adenosine stimulates blood vessel formation in the chick chorioallantoic membrane and embryo (Dusseau et al., 1986; Dusseau et al., 1988; Adair et aal., 1989). Moreover, chronic elevation of tissue adenosine concentrations, induced by the adenosine reuptake blocker dipyridamole (Tornling et al., 1978; Tornling et al., 1980a; Tornling et al., 1980b; Adolfsson et al., 1981; Tornling, 1982a; Tornling, 1982b; Adolfsson et al., 1982; Adolfsson, 1986a; Adolfsson, 1986b; Mattfeldt and Mall, 1983; Mall et al., 1987; Torry et al., 1992; Symons et al., 1993; Belardinelli et al., 2001), or long-term administration of adenosine and its analogs (Ziada et al., 1984; Hudlicka et al., 1986; Wothe et al., 2002) promotes capillary proliferation in the heart and skeletal muscles.

These effects of adenosine are mediated at least in part by stimulating the production of growth factors that facilitate new blood vessel formation from pre-existing fully differentiated endothelial cells in a process known as angiogenesis. The inventors have previously demonstrated that stimulation of A_{2B} adenosine receptors in various cell types results in upregulation of several pro-angiogenic factors including vascular endothelial growth factor, basic fibroblast growth factor, IL-8 and insulin-like factor-1 (Grant et al., 1999; Feoktistov et al., 2002; Zeng et al., 2003; Feoktistov et al., 2003). Other adenosine receptor subtypes have been also implicated in angiogenesis Feoktistov et al., 2003; Merighi et al., 2005; Desai et al., 2005).

In addition to angiogenesis, neovascularization can occur in a process known as vasculogenesis. EPCs are critical to this process and participate in the development of vascular networks by differentiating into mature endothelial cells. There is evidence that application of an A_{2A} adenosine receptor agonist CGS 21680 to experimental excisional wounds stimulates vasculogenesis in the early phase of wound healing (Montesinos et al., 2004). However, the role of adenosine in EPC homing to the sites of tissue injury or ischemia has not been studied.

### SUMMARY OF THE INVENTION

Thus, in accordance with the present invention, there is provided a method of promoting cell adhesion to vascular endothelium in a subject comprising (a) identifying a subject in need of tissue regeneration or neovascularization; (b) providing a cell expressing adenosine receptors; (c) contacting the cell with an adenosine receptor agonist; and (d) administering the cell to the subject. Alternatively, an adenosine receptor agonist, adenosine precursor or adenosine potentiating agent can be given before, together with, or after the cell to the subject. The subject may suffer from cardiovascular disease, and in particular, from cardiac ischemia or heart failure. The cell may be a stem cell, for example, stem-cell enriched or unfractionated preparations from bone marrow, mesenchymal or skeletal stem cells, or culture-expanded endothelial progenitor cells (EPCs). The stem cells may be autologous or heterologous to the subject. Step (d) may comprise antegrade infusion into coronary arteries or retrograde infusion via coronary sinus. The method may further comprise the step of obtaining the stem cells. Obtaining the stem cells may comprise tissue, bone marrow or peripheral blood collection and cell fractionation. The stem cells may be cultured prior to step (c). The adenosine (A₁ and A₂B) receptor agonist may be adenosine, its precursors (e.g., AMP), or a potentiator of endogenous adenosine (e.g., dipyridamole).

In another embodiment, there is provided a method of revascularizing an ischemic tissue in a subject comprising (a) providing a stem cell, such as an endothelial progenitor cell (EPC), expressing adenosine receptors; (b) contacting the cell with an adenosine ligand; and (c) administering the cell to the subject. The stem cells may be an EPC or a cardiac stem cell, or derived from bone marrow. The stem cell may be autologous or heterologous to the subject. The ischemic tissue may be cardiac tissue and step (c) may comprise intracardiac infusion or the stem cells. The method may further comprise the step of obtaining stem cells. Obtaining the stem cells may comprise tissue, bone marrow or peripheral blood collection and cell fractionation. The stem cells may be cultured prior to step (c). The adenosine (A₁ and A_{2B}) receptor agonist may be adenosine, its precursors (e.g., AMP), or a potentiator of endogenous adenosine (e.g., dipyridamole).

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.”

These, and other, embodiments of the invention will be better appreciated and understood when considered in conjunction with the following description and the accompanying drawings. It should be understood, however, that the following description, while indicating various embodiments of the invention and numerous specific details thereof, is given by way of illustration and not of limitation. Many substitutions, modifications, additions and/or rearrangements may be made within the scope of the invention without departing from the spirit thereof, and the invention includes all such substitutions, modifications, additions and/or rearrangements.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-D. Adenosine receptors in mouse eEPCs. FIG. 1A, Real-time RT-PCR analysis of mRNA encoding adenosine receptor subtypes. FIG. 1B, Effects of forskolin and NECA on cAMP accumulation. FIG. 1C, Effect of the selective A1 receptor agonist CPA on cAMP accumulation induced by 1 μmol/L forskolin. FIG. 1D, Effects of the selective A₁ receptor antagonists DPCPX and N-0861 on inhibition of forskolin-induced cAMP accumulation produced by 10 nmol/L CPA. The data are means±SEM (n=3).

FIGS. 2A-D. Adenosine receptors in MCEC-1 cells. FIG. 2A, Real-time RT-PCR analysis of mRNA encoding adenosine receptor subtypes. FIG. 2B, cAMP accumulation induced by the nonselective agonist NECA and the A2A selective agonist CGS21680. FIG. 2C, Effect of the selective A2B antagonist IPDX on NECA-induced cAMP accumulation. Concentration-response curves for NECA were repeated in the absence (open circles) and presence of 3 μmol/L (closed circles), 10 μmol/L (triangles), and 30 μmol/L (diamonds) IPDX. Inset, Schild analysis indicated simple competitive antagonism at A_{2B} receptors (slope of 1.1) with a KB value of 603 mmol/L. FIG. 2D, Effect of the selective A₁ receptor agonist CPA on cAMP accumulation induced by 1 μmol/L forskolin. The data are means±SEM (n=3).

FIGS. 3A-B. Adenosine receptors in HMVEC-c cells. FIG. 3A, Real-time RT-PCR analysis of mRNA encoding adenosine receptor subtypes. FIG. 3B, cAMP accumulation induced by the nonselective agonist NECA and the A₂A selective agonist CGS21680. The data are means±SEM (n=3).

FIGS. 4A-F. Adenosine receptor-mediated eEPC adhesion to MCEC-1 cells. FIG. 4A, Time course of the effect of 1 μmol/L NECA on eEPC adhesion to MCEC-1 cells. The data are presented as increases over basal adhesion in the absence of NECA at each time point. The data are means±SEM (n=12). FIG. 4B, Representative micrographs showing adhesion of fluorescently labeled eEPCs (green) to MCEC-1 monolayers in the absence (Basal) or presence of 1 μmol/L NECA. FIG. 4C, Effects of the nonselective adenosine agonist NECA, the selective A1 agonist CPA, and the selective A2A agonist CGS21680 on eEPC adhesion to MCEC-1 cells. The data are means±SEM (n=12 for NECA and CGS21680; n=24 for CPA). FIG. 4D, Effects of the selective A1 antagonists DPCPX and N-0861, the selective A_{2A} antagonist SCH58261, and the selective A2B antagonist IPDX on NECA-induced eEPC adhesion to MCEC-1 cells. The data are presented as percentages of an increase in adhesion induced by 1 μmol/L NECA. The data are means±SEM (n=12). FIG. 4E, Effect of pretreatment of eEPCs with pertussis toxin (PTX) compared with untreated cells (control) on their adhesion to MCEC-1 cells in the absence (basal) or presence of 10 μmol/L NECA. The data are means±SEM (n=6). *P<0.05 (t test) compared with control. FIG. 4F, Effect of NECA (10 μmol/L) on eEPC adhesion to MCEC-1 cells under defined flow conditions. The data are means±SEM (n=7). **P<0.01 (t test) compared with corresponding basal values.

FIG. 5. Adenosine receptor-mediated stimulation of adhesion of adult human EPCs to HMVEC-c cells. Effect of increasing concentrations of NECA on adhesion of human peripheral blood culture-expanded EPCs (CE-EPCs) to HMVEC-c cells. The data are mean±SEM of 3 separate cell preparations. *P<0.05, **P<0.01 (1-way ANOVA with Dunnett's post test).

FIGS. 6A-J. Adenosine promotes retention of eEPCs in isolated hearts. Retention of eEPCs in mouse hearts was studied using a conventional Langendorff retrograde perfusion system. FIGS. 6A-F, Representative fluorescent micrographs of perfused vessels (green) (FIGS. 6A and 6B), retained eEPCs (red) (FIGS. 6C and 6D), and their overlay (FIGS. 6E and 6F) were obtained from hearts perfused with eEPC suspension in the absence (FIGS. 6A, 6C, and 6E) or presence (FIGS. 6B, 6D, and 6F) of 10 μmol/L adenosine. (Scale bar=50 μm.) FIGS. 6G, 6I, and 6J, Retention of eEPCs in hearts perfused in the absence (Control) or presence of 10 μmol/L adenosine, 3 nmol/L CGS21680, and 100 μmol/L inosine was estimated by measuring the area of EPC-emitted fluorescence and by normalizing to the area of endothelial staining in 10 random images of the left ventricle taken for each heart. The data are means±SEM (n=3). *P<0.05 (t test). FIG. 6H, Concentration-response curves of CGS21680 and adenosine effects on coronary flow (mL/min per gram). The data are expressed as percentages from baseline and represent means±SEM (n=5).

FIGS. 7A-C. Interactions between PSGL-1 and P-selectin contribute to NECA-induced adhesion of eEPCs to MCEC-1 cells. FIG. 7A, Effect of fucoidan on eEPC adhesion to MCEC-1 cells in the absence (open bars) or in the presence of 10 μmol/L NECA (closed bars). The data are means±SEM (n=12). **P<0.01 (t test) compared with corresponding control values. FIG. 7B, Effect of blocking anti-PSGL-1 monoclonal antibody on eEPC adhesion to MCEC-1 cells in the absence (open bars) or presence (closed bars) of 10 μmol/L NECA. EPCs were preincubated for 15 min with a PSGL-1 blocking or control (rat IgG1) antibodies and then assayed for adhesion to MCEC-1 cells. The data are means±SEM (n=18). **P<0.01 (t test) compared with corresponding control values. FIG. 7C, Effect of NECA on cell surface P-selectin expression in MCEC-1 cells. Cells were incubated in the absence (open bars) or presence (closed bars) of 10 μmol/L NECA for 15 or 30 min at 37° C. Cell surface P-selectin expression was measured by an enzyme-linked immunoassay and presented in arbitrary units calculated from optical density of samples by subtracting corresponding values for nonspecific binding. The data are means±SEM (n=6)**P<0.01 (t test) compared with values obtained in the absence of NECA.

### DESCRIPTION OF ILLUSTRATIVE EMBODIMENTS

Neovascularization of ischemic tissues can be beneficial, if it promotes cell and tissue survival in patients suffering from cardiac ischemia, or may be detrimental if it supports tumor growth, development of retinopathy, and other pathological states. The formation of new capillaries to provide oxygen supply for ischemic tissues or tumors is a tightly regulated process that depends on a balance of pro-angiogenic and anti-angiogenic factors. In addition to the established view that angiogenic factors stimulate growth and migration of existing endothelial cells, there is also increasing evidence that bone marrow-derived circulating cells home to sites of ischemia and contribute directly or indirectly to neovascularization.

Hypoxia is a powerful inducer of neovascularization, and is also known to increase interstitial adenosine to levels that engage all adenosine receptors, including the low affinity A_{2B} receptor subtype. Affinity of A_{2B} receptors to adenosine is 30-80 times lower than affinities of other adenosine receptor subtypes (Fredholm et al., 2001). Due to this unique feature, A_{2B} receptors are likely to remain silent under normal physiological conditions. However, in pathophysiological situations associated with tissue hypoxia, A_{2B} receptors may become an integral part of a feedback mechanism aimed to restore oxygen supply to the affected tissues. Indeed, the inventors' recent studies revealed that A_{2B} receptors stimulate production of angiogenic factors and promote angiogenesis (Grant et al., 1999; Feoktistov et al., 2002; Feoktistov et al., 2003; Grant et al., 2001).

In the present study, the inventors tested the hypothesis that adenosine receptors can be also involved in homing of EPCs by increasing their adhesion to endothelium. They chose mouse embryonic EPCs and cardiac microvascular endothelial cell line MCEC-1 as a model to study the role of adenosine receptors in promoting adhesion. The choice of these cells was determined by their robust growth properties in culture, and hence availability of considerable quantities required for a systematic pharmacological analysis of adenosine receptors and their functions. These eEPCs express early endothelial markers, differentiate to mature endothelial cells, form vascular tubes in vitro, and build blood vessels after transplantation during embryogenesis (Hatzopoulos et al., 1998). In addition, the homing of eEPCs to hypoxic tumors, their participation in tumor vessel formation (Wei et al., 2004), as well as their stimulation of angiogenesis in chronic and acute ischemia (Kupatt et al., 2005) render these cells a relevant model system to study the biology of EPCs. Likewise, the conditionally immortalized MCEC-1, isolated from H-2 K_{b}-tsA58 transgenic mice containing a gene encoding the thermolabile SV40 T antigen, assume a phenotype virtually identical to that of primary cardiac microvascular endothelial cells when cultured at 37° C. (Lidington et al., 2002).

This study demonstrated that mouse eEPCs functionally express the high-affinity A₁ adenosine receptors, whereas the low-affinity A_{2B} is the predominant adenosine receptor subtype in MCEC-1. The inventors also detected the expression of mRNA encoding A_{2B} receptors in eEPCs and A₁ and A_{2A} receptors in MCEC-1. However, these experiments using specific adenosine receptor agonists and antagonists to modulate activity of adenylate cyclase argue against their significant functional role. The inventors verified that A_{2B} is also the predominant adenosine receptor subtype in primary cultured human cardiac microvascular endothelial cells, thus validating the use of MCEC-1 as a relevant cell model to study adenosine actions on cardiac microvascular endothelium.

Adenosine has been previously shown to modulate cell adhesion to vascular endothelium. Studies in neutrophils suggested differential roles of adenosine receptor subtypes in regulating their adhesion to endothelial cells. Stimulation of A₁ receptors promoted neutrophil adhesion to endothelial cells whereas stimulation of A_{2A} receptors inhibited their adhesion (Cronstein et al., 1992). The opposite roles of A₁ and A_{2A} receptors in regulation of neutrophil adherence to cardiac vascular endothelium were also demonstrated in the guinea pig isolated heart (Zahler et al., 1994). Endothelial A_{2A} receptors have been also shown to inhibit E-selectin and VCAM-1 expression in HUVECs activated by pro-inflammatory cytokines and endotoxin (Bouma et al., 1996). For this and other reasons, A_{2A} receptors are considered to mediate anti-inflammatory effects (Cronstein, 1994). Endothelial cells, however, are heterogeneous and exhibit variable patterns of adenosine receptors expression. Endothelial cells isolated from large vessels, e.g., HUVEC, predominantly express A_{2A} receptors and adenosine down-regulates pro-angiogenic and pro-inflammatory cytokines in these cells (Feoktistov et al., 2002; Bouma et al., 1996). In contrast, microvascular endothelial cells of various origins predominantly express A_{2B} receptors and respond to adenosine by secretion of pro-angiogenic factors (Grant et al., 1999; Feoktistov et al., 2002). Based on these observations, the inventors reasoned that, in contrast to the inhibitory action of A_{2A} receptors in neutrophil adhesion, A_{2B} receptors might promote adhesion of EPCs to microvascular endothelium.

In the present study, the inventors found that mouse eEPCs expressing A₁ receptors increased their adherence to MCEC-1 cells in the presence of the non-selective adenosine receptor agonist NECA under static and flow conditions. However, activation of A₁ receptors on eEPCs per se was not sufficient for efficient stimulation of cell adhesion; the effect of the selective A₁ agonist CPA was considerably lower than the effect of NECA. Pharmacological analysis of interactions between eEPCs and MCEC-1 indicated involvement of both A₁ and A_{2B} receptors in stimulation of cell adhesion. Furthermore, uncoupling of A₁ receptors from intracellular signaling pathways with pertussis toxin in eEPCs attenuated, but did not block completely the effect of NECA on their adhesion to MCEC-1. In contrast to neutrophils, the inventors found no evidence of A_{2A} receptor involvement in this process in agreement with the absence of functional expression of this adenosine receptor subtype in eEPCs and MCEC-1. Therefore, the data suggest that both A₁ receptors expressed on eEPCs and A_{2B} receptors expressed on MCEC-1 contribute to the increased interactions between these cells. It is possible that engagement of the high affinity A₁ receptors is especially important for circulating cells moving toward a gradient of adenosine concentrations generated by hypoxia, whereas the low affinity A_{2B} receptors are important for regulation of adhesive properties of endothelium located in the vicinity of the ischemic loci where concentrations of adenosine are the highest.

Upregulation of adhesion molecules by inflammatory cytokines and agents such as TNFα, IL-1β and LPS has been demonstrated in cardiac microvascular endothelial cells (Lidington et al., 2002). This process requires several hours to develop because it involves gene expression with subsequent protein synthesis de novo and the eventual increase in surface expression of adhesion molecules. These data, however, show that adenosine-dependent stimulation of EPC adhesion is a rapid process that reaches maximum within min. It is likely therefore, that adenosine regulates translocation of pre-existing adhesion molecules to the endothelial surface. It has been previously shown that the adhesion molecule P-selectin is stored in endothelial cell granules known as Weibel-Palade bodies. G protein-coupled receptors or substances that increase cAMP, intracellular Ca²⁺ or PKC activity can induce exocytosis of the content of Weibel-Palade bodies, thereby increasing P-selectin surface expression within min of stimulation (Tranquille and Emeis, 1991; Vischer and Wollheim, 1998; Cleato et al., 2005). The inventors have previously shown that A_{2B} adenosine receptors are linked to stimulation of these pathways in microvascular endothelial cells (Grant et al., 1999; Feoktistov et al., 2002), and in this study they found that stimulation of adenosine receptors for 15 or 30 min significantly increased P-selectin expression on MCEC-1 surface, suggesting that this mechanism may be relevant to the rapid increase of EPC adhesion to cardiac microvascular endothelial cells induced by adenosine. This observation is in agreement with the rapid recruitment of circulating EPCs observed in myocardial ischemia. It has been reported that circulating EPCs are immediately recruited to myocardium within the first hour from the onset of ischemia in a mouse model (Ii et al., 2005) and adenosine is known to be released into the coronary circulation within min from the onset of ischemia (Chlopicki et al., 1998).

Mouse eEPCs express a wide range of adhesion molecules on their surface that potentially can interact with their counterparts on endothelial cells (Vajkoczy et al., 2003). In particular, the P-selectin ligand PSGL-1 has been suggested to play an important role in adhesion of these cells to the vascular wall (Vajkoczy et al., 2003; Langer et al., 2006). In this study, the inventors found that the P/L selectin inhibitor fucoidan and a blocking antibody against PSGL-1, attenuated the NECA-induced adhesion of eEPCs to MCEC-1. This inhibition, however, was partial suggesting that multiple adhesion molecules may be involved in this process.

To evaluate if the central findings of this study, derived from a murine progenitor cell model, can be applied to human progenitor cells, key experiments were performed using two additional cell types, namely adult human peripheral blood CD34⁺ cells and culture-expanded EPCs. Indeed, the inventors found that stimulation of adenosine receptors increased adhesion of human progenitor cells to human cardiac microvascular endothelial cells. These results, obtained in murine and human cells, may have important implications not only for the understanding of molecular mechanisms of neovascularization, but also for a novel therapeutic use of adenosine. There is growing interest in cell-based therapeutic approaches to improve vascularization of ischemic organs, including the heart. One of the major problems for the therapeutic use of EPCs is that a majority of injected cells passes through the targeted organ (e.g., heart) and accumulates in other organs such as spleen, liver and kidney (Aicher et al., 2003). In this study, the inventors demonstrated that adenosine promotes EPC retention in vasculature of isolated hearts suggesting its potential use for improvement of cell delivery. Adenosine can be given directly into the coronary circulation, and its extremely short half-life in the bloodstream provides the unique advantage of increasing EPC retention locally. Intracoronary adenosine has been administered in humans to induce preconditioning without significant adverse events (Leesar et al., 1997; Marzilli et al., 2000; Leesar et al., 2003). Recent studies showed that retrograde infusion of EPCs through the coronary sinus significantly improves their retention in the heart (Kupatt et al., 2005). The data suggest that adenosine could further improve delivery of progenitor cells by increasing their adhesion to cardiac endothelium, a particularly appealing prospect due to the clinical availability of adenosine.

### I. CARDIOVASCULAR DISEASE

Cardiovascular diseases are among the most common natural causes of death. The cardiovascular diseases include many serious diseases which involve the cardiac and vascular systems, such as atherosclerosis, ischemic heart diseases, cardiac failure, cardiac shock, arrhythmia, hypertension, cerebral vascular diseases and peripheral vascular diseases.

Atherosclerosis most often occurs as a complication of hyperlipidemia and can be treated with antihyperlipidemic agents. Ischemic heart disease, cardiac failure, cardiac shock, cerebral vascular disease, peripheral vascular disease, hypertension, arrhythmia and arteriosclerosis may be fatal because ischemia develops in various organs such as the heart, brain and the walls of blood vessels. The ischemia damages the organs in which it develops because it impairs the functions of mitochondria that produce adenosine triphosphate (ATP), which is a phosphate compound with high energy potential serving as an energy source for the constituent cells of these organs. The resulting functional damage of organs can be fatal if it occurs in vital organs such as the heart, brain and blood vessels. It is therefore important for treating these diseases to restore the functional impairment of mitochondria caused by ischemia. Antiarrhythmic agents have been used to treat ischemic heart disease and arrhythmia, but their use with patients with possible cardiac failure has been strictly limited because these agents may cause cardiac arrest by their cardiodepressant effects.

The cardiovascular diseases named above may develop independently, but more often than not they occur in various combinations. For example, ischemic heart diseases are frequently accompanied by arrhythmia and cardiac failure, and complications of cerebrovascular disorder with hypertension are well known. Atherosclerosis is often complicated by one or more cardiovascular diseases and can make the patient seriously ill.

Cardiovascular diseases, which are often complicated by other cardiovascular diseases, have often been treated with a combination of multiple drugs, each of which is specific for a single disease. However, drug-therapy employing multiple agents presents problems for both doctors and patients: doctors always consider compatibilities and contraindications of drugs, and patients suffer both mental and physical distresses due to complicated administration of various drugs and high incidence of adverse reactions. Therefore, it has long been desired to develop a therapeutic agent that has overall pharmacological activities against cardiovascular diseases and which can be employed in the treatment of these diseases with high efficacy.

A. DCM and Chronic Heart Failure

As discussed above, cardiovascular diseases encompass a huge array of syndromes and disorders, all of which combined are among the leading causes of death worldwide. Heart failure by itself is one of the leading causes of morbidity and mortality in the world. In the U.S. alone, estimates indicate that 3 million people are currently living with cardiomyopathy and another 400,000 are diagnosed on a yearly basis. Dilated cardiomyopathy (DCM), also referred to as “congestive cardiomyopathy,” is the most common form of the cardiomyopathies and has an estimated prevalence of nearly 40 per 100,000 individuals (Durand et al., 1995). Although there are other causes of DCM, familiar dilated cardiomyopathy has been indicated as representing approximately 20% of “idiopathic” DCM. Approximately half of the DCM cases are idiopathic, with the remainder being associated with known disease processes. For example, serious myocardial damage can result from certain drugs used in cancer chemotherapy (e.g., doxorubicin and daunoribucin), or from chronic alcohol abuse. Peripartum cardiomyopathy is another idiopathic form of DCM, as is disease associated with infectious sequelae. In sum, cardiomyopathies, including DCM, are significant public health problems.

Heart disease and its manifestations, including coronary artery disease, myocardial infarction, congestive heart failure and cardiac hypertrophy, clearly present a major health risk in the United States today. The cost to diagnose, treat and support patients suffering from these diseases is well into the billions of dollars. Two particularly severe manifestations of heart disease are myocardial infarction and cardiac hypertrophy. With respect to myocardial infarction, typically an acute thrombocytic coronary occlusion occurs in a coronary artery as a result of atherosclerosis and causes myocardial cell death. Because cardiomyocytes, the heart muscle cells, are terminally differentiated and generally incapable of cell division, they are generally replaced by scar tissue when they die during the course of an acute myocardial infarction. Scar tissue is not contractile, fails to contribute to cardiac function, and often plays a detrimental role in heart function by expanding during cardiac contraction, or by increasing the size and effective radius of the ventricle, for example, becoming hypertrophic.

As mentioned above, treatment with pharmacological agents still represents the primary mechanism for reducing or eliminating the manifestations of heart failure. Diuretics constitute the first line of treatment for mild-to-moderate heart failure. Unfortunately, many of the commonly used diuretics (e.g., the thiazides) have numerous adverse effects. For example, certain diuretics may increase serum cholesterol and triglycerides. Moreover, diuretics are generally ineffective for patients suffering from severe heart failure.

If diuretics are ineffective, vasodilatory agents may be used; the angiotensin converting (ACE) inhibitors (e.g., enalopril and lisinopril) not only provide symptomatic relief, they also have been reported to decrease mortality (Young et al., 1989). Again, however, the ACE inhibitors are associated with adverse effects that result in their being contraindicated in patients with certain disease states (e.g., renal artery stenosis). Similarly, inotropic agent therapy (i.e., a drug that improves cardiac output by increasing the force of myocardial muscle contraction) is associated with a panoply of adverse reactions, including gastrointestinal problems and central nervous system dysfunction.

The currently used pharmacological agents have severe shortcomings in particular patient populations. The availability of new, safe and effective agents would undoubtedly benefit patients who either cannot use the pharmacological modalities presently available, or who do not receive adequate relief from those modalities. The prognosis for patients with DCM is variable, and depends upon the degree of ventricular dysfunction, with the majority of deaths occurring within five years of diagnosis.

B. Myocardial Infarction and Ischemic Heart Disease

Ischemic heart disease is the leading cause of death in industrialized countries. The management of ischemic heart disease essentially relies upon one of three strategies, comprising medical therapy, percutaneous transluminal procedures, such as coronary angioplasty and atherectomy, and coronary artery bypass grafting. Although medical treatment remains the mainstay of anti-ischemic therapy, many patients undergo additional, invasive therapy in an attempt to restore coronary blood flow. However, there is increasingly intense discussion regarding not only the relative merits of these therapeutic approaches but also the point within the management of ischaemic heart disease at which they should be applied and the type of patient for which each is more appropriate.

Acute myocardial infarction (MI) strikes the majority of sufferers without prior warning and in the absence of clinically detectable predisposing risk factors (for a full review, see Braunwald, 1997). When patients come to the intensive unit in a hospital showing symptoms of acute MI, the diagnosis for acute MI requires that the patients must have (1) an increase in the plasma concentration of cardiac enzymes and (2) either a typical clinical presentation and/or typical ECG changes. Either of the following parameters will fulfill the requirement for an increase in cardiac enzymes: (1) Total creatine-kinase (CK) at least 2 times the upper limit of the normal range, or (2) CK-MB (muscle-brain) above the upper limit of the normal range and at least 5% of the normal CK. If total CK or CK-MB is not available, the following will be accepted in the fulfillment of the criteria for acute MI: (1) Troponin T at least 3 times the upper limit of the normal range; (2) Troponin I at least 3 times the upper limit of the normal range. The use of Troponin T as a serum marker for MI is disclosed in Murthy and Karmen (1997). The analytical performance and clinical utility of a sensitive immunoassay for determination of cardiac Troponin I can be taken from Davies et al. (1997).

Typical ECG changes include evolving ST-segment or T-wave changes in two or more contiguous ECG leads, the development of new pathological Q/QS waves in two or more contiguouos ECG leads, or the development of new left bundle branch block.

Secondary prevention, namely the implementation of therapy to postpone further coronary events, thus continues to remain the major goal of prophylactic drug therapy in these patients. Survivors of acute MI are at moderate risk of recurrent infarction or cardiac death. Morbidity and mortality following an MI may be related to arrhythmias, to left ventricular dysfunction, and to recurrent MI. Because aspirin had a significant protective effect in secondary prevention of vascular disease, the possible benefit of aspirin in primary prevention was tested. However, several studies have shown that only a limited percent of the population at risk really benefits from aspirin therapy (Cairns et al., 1995). Thus, while the concept of secondary prevention of reinfarction and death after recovery from an MI has been actively investigated for several decades, there have been problems in proving the efficacy of various interventions. These problems have been related both to the ineffectiveness of certain strategies and to the difficulty in proving a benefit as mortality and morbidity have improved following MI.

The development of the AT (1) receptor antagonists provided, in addition to the ACE inhibitors, a new, more specific pharmacological tool to inhibit the renin-angiotensin cascade. However, there are distinguishing features between AT (1) receptor antagonists and ACE inhibitors that highlight their current limitations. One is manifested by the concomitant potentiation of bradykinin produced by ACE inhibitors, since the kinase II and converting enzyme are one in the same. The bradykinin related mechanism mediated through nitric oxide, prostaglandins, and endothelially derived hyper-polaring factor may be responsible for a different clinical effect of ACE inhibitors. Furthermore, the effect of the AT (2) is not yet clear, as an inhibition of the AT (1) receptor leads to an increase of AT (2).

### II. ADENOSINE AND ADENOSINE RECEPTOR AGONISTS

A. Adenosine

Adenosine is a nucleoside comprised of adenine attached to a ribose (ribofuranose) moiety via a β-N⁹-glycosidic bond. Adenosine plays an important role in biochemical processes, such as energy transfer—as adenosine triphosphate (ATP) and adenosine diphosphate (ADP)— as well as in signal transduction as cyclic adenosine monophosphate (cAMP). It is also an inhibitory neurotransmitter, believed to play a role in promoting sleep and suppressing arousal, with levels increasing with each hour an organism is awake.

Adenosine is an endogenous purine nucleoside that modulates many physiologic processes. Cellular signaling by adenosine occurs through four known adenosine receptor subtypes (A₁, A_{2A}, A_{2B}, and A₃), all of which are seven transmembrane spanning G-protein coupled receptors. These four receptor subtypes are further classified based on their ability to either stimulate or inhibit adenylate cyclase activity. The A_{2A} and A_{2B} receptors couple to Gs and mediate the stimulation of adenylate cyclase, while the A₁ and A₃ adenosine receptors couple to Gi which inhibits adenylate cyclase activity. Additionally, A₁ receptors couple to Go, which has been reported to mediate adenosine inhibition of Ca²⁺ conductance, whereas A_{2B} and A₃ receptors also couple to Gq and stimulate phospholipase activity Extracellular adenosine concentrations from normal cells are approximately 300 nM; however, in response to cellular damage (e.g., in inflammatory or ischemic tissue), these concentrations are quickly elevated (600-1,200 mM). Thus, in regards to stress or injury, the function of adenosine is primarily that of cytoprotection preventing tissue damage during instances of hypoxia, ischemia, and seizure activity. Activation of A_{2A} receptors produces a constellation of responses that in general can be classified as anti-inflammatory.

Adenosine is a potent anti-inflammatory agent, acting at its four G-protein coupled receptors. Topical treatment of adenosine to foot wounds in diabetes mellitus has been shown in lab animals to drastically increase tissue repair and reconstruction. Topical administration of adenosine for use in wound healing deficiencies and diabetes mellitus in humans is currently under clinical investigation.

When administered intravenously, adenosine causes transient heart block in the AV node. It also causes endothelial dependent relaxation of smooth muscle as is found inside the artery walls. This causes dilatation of the “normal” segments of arteries where the endothelium is not separated from the tunica media by atherosclerotic plaque. This feature allows physicians to use adenosine to test for blockages in the coronary arteries, by exaggerating the difference between the normal and abnormal segments.

In individuals suspected of suffering from a supraventricular tachycardia (SVT), adenosine is used to help identify the rhythm. Certain SVTs can be successfully terminated with adenosine. This includes any re-entrant arrhythmias that require the AV node for the re-entry (e.g., AV reentrant tachycardia (AVRT), AV nodal reentrant tachycardia (AVNRT). In addition, atrial tachycardia can sometimes be terminated with adenosine.

Adenosine has an indirect effect on atrial tissue causing a shortening of the refractory period. When administered via a central lumen catheter, adenosine has been shown to initiate atrial fibrillation because of its effect on atrial tissue. In individuals with accessory pathways, the onset of atrial fibrillation can lead to a life threatening ventricular fibrillation.

Fast rhythms of the heart that are confined to the atria (e.g., atrial fibrillation, atrial flutter) or ventricles (e.g., monomorphic ventricular tachycardia) and do not involve the AV node as part of the re-entrant circuit are not typically converted by adenosine, however, the ventricular response rate will be temporarily slowed.

Because of the effects of adenosine on AV node-dependent SVTs, adenosine is considered a class V anti-arrhythmic agent. When adenosine is used to cardiovert an abnormal rhythm, it is normal for the heart to enter asystole for a very brief period. While the adenosine is necessary to save to patient, the event of the heart stopping for several seconds is very disconcerting to a normally conscious patient. This effect of temporary arrest is often overlooked and not mentioned, except in professional medical literature.

The pharmacological effects of adenosine are blunted in individuals who are taking methylxanthines (e.g., caffeine and theophylline). Caffeine's stimulatory effects are primarily (although not entirely) credited to its inhibition of adenosine by binding to the same receptors. By nature of caffeine's purine structure it binds to some of the same receptors as adenosine, effectively blocking adenosine receptors in the central nervous system. This reduction in adenosine activity leads to increased activity of the neurotransmitters dopamine and glutamate.

When given for the evaluation or treatment of an SVT, the initial dose is 6 mg, given as a fast IV/Intraosseous IO push. Due to adenosine's extremely short half-life, start the IV line as proximal to the heart as possible, such as the antecubital fossa. It is also recommended to follow the IV push with an immediate flush of 5-10 ccs of saline. If this has no effect (e.g., no evidence of transient AV block), a 12 mg dose can be given 1-2 min after the first dose. If the 12 mg dose has no effect, a second 12 mg dose can be administered 1-2 min after the previous dose. Some clinicians may prefer to administer a higher dose (typically 18 mg), rather than repeat a dose that apparently had no effect. When given to dilate the arteries, such as in a “stress test,” the dosage is typically 0.14 mg/kg/min, administered for 4 or 6 min, depending on the protocol.

Beta blockers and dopamine may precipitate toxicity in the patient when given at the same time as adenosine. Contraindications include tachycardia, a (relative contraindication), 2nd or 3rd degree heart block, atrial fibrillation, atrial flutter, ventricular tachycardia, cick sinus syndrome, Stokes-Adams Attack, Wolf-Parkinson-White syndrome, and bradycardia with Premature Ventricular Contractions (PVCs). Many individuals experience facial flushing, lightheadedness, diaphoresis, or nausea after administration of adenosine. These symptoms are transitory, usually lasting less than one minute.

B. Adenosine Precursors

Adenosine monophosphate, also known as 5′-adenylic acid and abbreviated AMP, is a nucleotide that is found in RNA. It is an ester of phosphoric acid with the nucleoside adenosine. AMP consists of the phosphate group, the pentose sugar ribose, and the nucleobase adenine. AMP can also exist as a cyclic structure known as cyclic AMP (or cAMP). Within certain cells, the enzyme adenylate cyclase makes cAMP from ATP, and typically this reaction is regulated by hormones such as adrenaline or glucagon. cAMP plays an important role in intracellular signaling. AMP is dephosphorylated by ecto-5′-nucleotidase, producing adenosine under hypoxic conditions. Other precursors of adenosine include ATP and ADP.

C. Adenosine Potentiators

Adenosine has a very short half-live when infused in to humans because of uptake into blood cells and tissue cells. This uptake mechanism can be blocked by adenosine reuptake blockers such as dipyridamole and others. Adenosine is also destroyed by enzymatic metabolism with adenosine deaminase. This enzyme can be inhibited by EHNA and other inhibitors. Adenosine reuptake inhibitors, and inhibitors of the enzymatic degradation of adenosine, therefore, can be used as potentiators of adenosine actions.

D. AR Agonists

Selective A1 agonists agonists are well-known in the art. Among them are R-PIA, CPA and CCPA. Other useful adenosine receptor agonists, in particular those with selectivity for the A₂ receptor are well-known in the art. These include 2-substituted adenosine-5′-carboxamide derivatives (U.S. Pat. Nos. 4,968,697 and 5,034,381) and N9 cyclopentyl-substituted adenine derivative (U.S. Pat. No. 5,063,233). Particular selective A_{2A} agonists include CGS21680 (2-p-(carboxyethyl)phenethylamino-5′-N-ethylcarbox-amidoadenosine), DPMA (N⁶-[2-(3,5-dimethoxyphenyl)-2-(2-methylphenyl)]ethyl adenosine), HENECA, CV-1808, CV-1674, and ATL146e. Particular A_{2B} agonist include NECA (5′-N-ethylcarboxamido-adenosine), (S)—PHPNECA((S)-2-phenylhydroxypropynylNECA and 2-amino-pyridine-3,5-dicarbonitrile derivatives, and N(6)-[(hetero)aryl/(cyclo)alkyl-carbamoyl-methoxy-phenyl]-(2-chloro)-5′-N-ethylcarboxamido-adenosines. Additional AR agonists for use in the present invention are described in Lambertucci et al. (2003); Vittori et al. (2004); Beukers et al. (2004); Baraldi et al. (2007), incorporated herein by reference.

### III. STEM CELLS

Stem cells are primal cells found in all multi-cellular organisms that retain the ability to renew themselves through mitotic cell division and can differentiate into a diverse range of specialized cell types. The three broad categories of mammalian stem cells are embryonic stem cells (derived from blastocysts), adult stem cells (found in adult tissues), and cord blood stem cells (found in the umbilical cord). In a developing embryo, stem cells can differentiate into all of the specialized embryonic tissues. In adult organisms, stem cells and progenitor cells act as a repair system for the body, replenishing specialized cells.

As stem cells can be readily grown and transformed into specialised cells with characteristics consistent with cells of various tissues such as muscles or nerves through cell culture, their use in medical therapies has been proposed. In particular, embryonic cell lines, autologous embryonic stem cells generated through therapeutic cloning, and highly plastic adult stem cells from the umbilical cord blood or bone marrow are touted as promising candidates.

A. Embryonic Stem (ES) Cells

ES cells are cells derived from the epiblast tissue of the inner cell mass (ICM) of a blastocyst. A blastocyst is an early stage embryo—approximately 4 to 5 days old in humans and consisting of 50-150 cells. ES cells are pluripotent, and give rise during development to all derivatives of the three primary germ layers: ectoderm, endoderm and mesoderm. In other words, they can develop into each of the more than 200 cell types of the adult body when given sufficient and necessary stimulation for a specific cell type. They do not contribute to the extra-embryonic membranes or the placenta.

A human embryonic stem cell is defined by the presence of several transcription factors and cell surface proteins. The transcription factors Oct-4, Nanog, and Sox2 form the core regulatory network which ensures the suppression of genes that lead to differentiation and the maintenance of pluripotency. The cell surface proteins most commonly used to identify hES cells are the glycolipids SSEA3 and SSEA4 and the keratan sulfate antigens Tra-1-60 and Tra-1-81. The molecular definition of a stem cell includes many more proteins and continues to be a topic of research.

B. Adult Stem Cells

Adult stem cells are undifferentiated cells found throughout the body that divide to replenish dying cells and regenerate damaged tissues. Also known as somatic stem cells, they can be found in children, as well as adults. A great deal of adult stem cell research has focused on clarifying their capacity to divide or self-renew indefinitely and their differentiation potential. Adult stem cells, like embryonic stem cells, have pluripotent potential and can differentiate into cells derived from all three germ layers. In mice, pluripotent stem cells can be directly generated from adult fibroblast cultures.

While embryonic stem cell potential remains untested, adult stem cell treatments have been used for many years to successfully treat leukemia and related bone/blood cancers through bone marrow transplants. The use of adult stem cells in research and therapy is not as controversial as embryonic stem cells, because the production of adult stem cells does not require the destruction of an embryo.

C. Endothelial Progenitor Cells

Endothelial progenitor cells are bone marrow-derived cells that circulate in the blood and have the ability to differentiate into endothelial cells, the cells that make up the lining of blood vessels. The process by which blood vessels are born de novo from endothelial progenitor cells is known as vasculogenesis. Most of vasculogenesis occurs in utero during embryologic development. Endothelial progenitor cells found in adults are thus related to angioblasts, which are the stem cells that form blood vessels during embryogenesis. Endothelial progenitor cells are thought to participate in pathologic angiogenesis such as that found in retinopathy and tumor growth. While angioblasts have been known to exist for many years, adult endothelial progenitor cells were only characterized in the 1990's after Asahara and colleagues published that a purified population of CD34-expressing cells isolated from the blood of adult mice could differentiate into endothelial cells in vitro. As endothelial progenitor cells are originally derived from the bone marrow, it is thought that various cytokines, growth factors, and hormones cause them to be mobilized from the bone marrow and into the peripheral circulation where they ultimately are recruited to regions of angiogenesis.

In animal models of myocardial infarction, the injection of ex vivo expanded EPCs or stem and progenitor cells significantly improved blood flow and cardiac function and reduced left ventricular scarring. Similarly, infusion of ex vivo expanded EPCs deriving from peripheral blood mononuclear cells in nude mice or rats improved the neovascularization in hind limb ischemia models. Correspondingly, initial pilot trials indicate that bone marrow-derived or circulating blood-derived progenitor cells are useful for therapeutically improving blood supply of ischemic tissue. In addition, transplantation of ex vivo expanded EPCs significantly improved coronary flow reserve and left ventricular function in patients with acute myocardial infarction.

Of the three cell markers (CD133, CD34, and the vascular endothelial growth factor receptor 2) that characterize the early functional EPCs in adult bone marrow, EPCs lose CD133/CD34 and start to express CD31, vascular endothelial cadherin, and von Willebrand factor when migrating to the circulation. Various isolation procedures of EPCs from different sources by using adherence culture or affinity magnetic microbeads have been described (e.g., WO/2005/078073; Asahara et al. (1997); Asahara et al. (1999); Hristov et al. (2003); Shaw et al. (2004); Werner et al. (2005).

### IV. METHODS OF TREATMENT

A. Existing Therapies

Heart disease of some forms may curable and these are dealt with by treating the primary disease, such as anemia or thyrotoxicosis. Also curable are forms caused by anatomical problems, such as a heart valve defect. These defects can be surgically corrected. However, for the most common forms of heart failure—those due to damaged heart muscle—no known cure exists. Treating the symptoms of these diseases helps, and some treatments of the disease have been successful. The treatments attempt to improve patients' quality of life and length of survival through lifestyle change and drug therapy. Patients can minimize the effects of heart failure by controlling the risk factors for heart disease, but even with lifestyle changes, most heart failure patients must take medication, many of whom receive two or more drugs.

Several types of drugs have proven useful in the treatment of heart failure: Diuretics help reduce the amount of fluid in the body and are useful for patients with fluid retention and hypertension; and digitalis can be used to increase the force of the heart's contractions, helping to improve circulation. Results of recent studies have placed more emphasis on the use of ACE inhibitors (Manoria and Manoria, 2003). Several large studies have indicated that ACE inhibitors improve survival among heart failure patients and may slow, or perhaps even prevent, the loss of heart pumping activity (for a review see De Feo et al., 2003; DiBianco, 2003).

Patients who cannot take ACE inhibitors may get a nitrate and/or a drug called hydralazine, each of which helps relax tension in blood vessels to improve blood flow (Ahmed, 2003).

Heart failure is almost always life-threatening. When drug therapy and lifestyle changes fail to control its symptoms, a heart transplant may be the only treatment option. However, candidates for transplantation often have to wait months or even years before a suitable donor heart is found. Recent studies indicate that some transplant candidates improve during this waiting period through drug treatment and other therapy, and can be removed from the transplant list (Conte et al., 1998).

Transplant candidates who do not improve sometimes need mechanical pumps, which are attached to the heart. Called left ventricular assist devices (LVADs), the machines take over part or virtually all of the heart's blood-pumping activity. However, current LVADs are not permanent solutions for heart failure but are considered bridges to transplantation.

As a final alternative, there is an experimental surgical procedure for severe heart failure available called cardiomyoplasty (Dumcius et al., 2003). This procedure involves detaching one end of a muscle in the back, wrapping it around the heart, and then suturing the muscle to the heart. An implanted electric stimulator causes the back muscle to contract, pumping blood from the heart. To date, none of these treatments have been shown to cure heart failure, but can at least improve quality of life and extend life for those suffering this disease.

As with heart failure, there are no known cures to hypertrophy. Current medical management of cardiac hypertrophy, in the setting of a cardiovascular disorder includes the use of at least two types of drugs: inhibitors of the rennin-angiotensoin system, and β-adrenergic blocking agents (Bristow, 1999). Therapeutic agents to treat pathologic hypertrophy in the setting of heart failure include angiotensin II converting enzyme (ACE) inhibitors and β-adrenergic receptor blocking agents (Eichhorn & Bristow, 1996). Other pharmaceutical agents that have been disclosed for treatment of cardiac hypertrophy include angiotensin II receptor antagonists (U.S. Pat. No. 5,604,251) and neuropeptide Y antagonists (PCT Publication No. WO 98/33791).

Non-pharmacological treatment is primarily used as an adjunct to pharmacological treatment. One means of non-pharmacological treatment involves reducing the sodium in the diet. In addition, non-pharmacological treatment also entails the elimination of certain precipitating drugs, including negative inotropic agents (e.g., certain calcium channel blockers and antiarrhythmic drugs like disopyramide), cardiotoxins (e.g., amphetamines), and plasma volume expanders (e.g., nonsteroidal anti-inflammatory agents and glucocorticoids).

B. Administration of AR Agonists

Administration of AR agonists will follow the protocols described for these agents in the respective literature. U.S. Pat. Nos. 4,968,697; 5,034,381; and 5,063,233. Generally, these drugs will be administered intravenously, i.e., systemically, but may be administered more or less locally, i.e., to the vasculature of the relevant region, such as ischemic heart tissue. Administration may be by continuous infusion, for example, using a portable pump, or by a series of bolus injections. Administration may be discontinued and restarted if side effects occur. Adenosine can be given via a catheter directly placed in the coronary arteries as an antegrade infusion, or can be given as a retrograde infusion via a catheter placed in the coronary sinus.

C. Ex vivo Cell-Based Therapy

In another embodiment, the present invention contemplates the use of stem cells, including EPCs, in particular from the subject to be treated, where those cells have been contacted ex vivo with an AR agonist such as adenosine or AMP. Culturing of EPCs will be performed according to standard methods described in the literature for expansion of EPC populations. At present, the bone marrow appears the most realistic source of stem cells to treat cardiomyopathies because it can provide both high numbers of progenitor cells, as well as cell diversity and the rich secretome likely required for clinical benefits.

Bone marrow cells can be obtained patients using standard protocols, and can be cultured ex vivo to enrich stem cells. Before readministration of cells, they may be incubated with adenosine or an [A₁] adenosine receptor agonist for 10-30 min. Cells are then administered intravenously, into the coronary arteries or by direct injection into the myocardium, to a patient in need of revasculartization or cell regeneration.

Readministration of stimulated EPCs will involve the following steps. (a) Administration of adenosine and then (b) cells together or without adenosine via antegrade infusion into coronary arteries or retrograde infusion via coronary sinus. Alternatively, cells together with adenosine may be infused without prior infusion of adenosine.

As an example, a patient will be taken to the catheterization laboratory. Femoral artery and vein accesses will be obtained. Heparin will be given to reach ACT>200. A left coronary artery guiding catheter will be used to engage the left main coronary artery. A 0.014″ Choice Floppy wire will be inserted to distal left anterior descending artery. After coronary angiography to determine the vessel size, an appropriate sized Voyager OTW Coronary Balloon Dilation Catheter will be advanced to the mid-left anterior descending artery (LAD) over the wire. A JR4 guide will be used to engage coronary sinus through the venous access. A 0.014″ Choice Floppy wire will be inserted to the medium coronary vein. After coronary sinus angiography to determine the size of the vessel, an appropriate sized Voyager balloon will be placed into the coronary sinus to temporally occlude the coronary sinus. Balloon catheter in LAD will be inflated to obstruct the forward flow (confirmed by angiography). Inflation pressure will be <4AIM—to minimize vascular injury. After balloon inflation, adenosine will be injected directly into a coronary artery at doses, for example of 24 mg over 140 min. Following this step, stem cells/EPCs will be infused directly into the LAD over 4 min.

D. Combined Therapy

In another embodiment, it is envisioned to use AR agonists or stimulated EPCs in combination with other therapeutic modalities. Thus, in addition to the therapies described above, one may also provide to the patient more “standard” pharmaceutical cardiac therapies. Examples of other therapies include, without limitation, so-called “beta blockers,” anti-hypertensives, cardiotonics, anti-thrombotics, vasodilators, hormone antagonists, iontropes, diuretics, endothelin antagonists, calcium channel blockers, phosphodiesterase inhibitors, ACE inhibitors, angiotensin type 2 antagonists and cytokine blockers/inhibitors, and HDAC inhibitors.

Combinations may be achieved by contacting cardiac cells with a single composition or pharmacological formulation that includes both agents, or by contacting the cell with two distinct compositions or formulations, at the same time, wherein one composition includes the expression construct and the other includes the agent. Alternatively, the therapy using AR agonists or stimulated EPCs may precede or follow administration of the other agent(s) by intervals ranging from minutes to weeks. In embodiments where the other agent and expression construct are applied separately to the cell, one would generally ensure that a significant period of time did not expire between the time of each delivery, such that the agent and expression construct would still be able to exert an advantageously combined effect on the cell. In such instances, it is contemplated that one would typically contact the cell with both modalities within about 12-24 hours of each other and, more preferably, within about 6-12 hours of each other, with a delay time of less than about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either AR agonists or stimulated EPCs, or the other agent will be desired. In this regard, various combinations may be employed. By way of illustration, where AR agonists or stimulated EPCs is “A” and the other agent is “B,” the following permutations based on 3 and 4 total administrations are exemplary:

A/B/A B/A/B B/B/A A/A/B B/A/A A/B/B B/B/B/A
B/B/A/B A/A/B/B A/B/A/B A/B/B/A B/B/A/A B/A/B/A
B/A/A/B B/B/B/A A/A/A/B B/A/A/A A/B/A/A A/A/B/A
A/B/B/B B/A/B/B B/B/A/B

Other combinations are likewise contemplated. Pharmacological therapeutic agents and methods of administration, dosages, etc., are well known to those of skill in the art (see for example, the “Physicians Desk Reference,” Goodman & Gilman's “The Pharmacological Basis of Therapeutics,” “Remington's Pharmaceutical Sciences,” and “The Merck Index, Thirteenth Edition,” incorporated herein by reference in relevant parts), and may be combined with the invention in light of the disclosures herein. Some variation in dosage will necessarily occur depending on the condition of the subject being treated. The person responsible for administration will, in any event, determine the appropriate dose for the individual subject, and such individual determinations are within the skill of those of ordinary skill in the art.

Non-limiting examples of a pharmacological therapeutic agent that may be used in conjunction with therapies of the present invention include an antihyperlipoproteinemic agent, an antiarteriosclerotic agent, an antithrombotic/fibrinolytic agent, a blood coagulant, an antiarrhythmic agent, an antihypertensive agent, a vasopressor, an antianginal agent, an antibacterial agent or a combination thereof. The following are exemplary of such combinations.
1. Antihyperlipoproteinemics

In certain embodiments, administration of an agent that lowers the concentration of one of more blood lipids and/or lipoproteins, known herein as an “antihyperlipoproteinemic,” may be combined with a cardiovascular therapy according to the present invention, particularly in treatment of athersclerosis and thickenings or blockages of vascular tissues. In certain aspects, an antihyperlipoproteinemic agent may comprise an aryloxyalkanoic/fibric acid derivative, a resin/bile acid sequesterant, a HMG CoA reductase inhibitor, a nicotinic acid derivative, a thyroid hormone or thyroid hormone analog, a miscellaneous agent or a combination thereof.
a. Aryloxyalkanoic Acid/Fibric Acid Derivatives

Non-limiting examples of aryloxyalkanoic/fibric acid derivatives include beclobrate, enzafibrate, binifibrate, ciprofibrate, clinofibrate, clofibrate (atromide-S), clofibric acid, etofibrate, fenofibrate, gemfibrozil (lobid), nicofibrate, pirifibrate, ronifibrate, simfibrate and theofibrate.
b. Resins/Bile Acid Sequesterants

Non-limiting examples of resins/bile acid sequesterants include cholestyramine (cholybar, questran), colestipol (colestid) and polidexide.
c. HMG CoA Reductase Inhibitors

Non-limiting examples of HMG CoA reductase inhibitors include lovastatin (mevacor), pravastatin (pravochol) or simvastatin (zocor).
d. Nicotinic Acid Derivatives

Non-limiting examples of nicotinic acid derivatives include nicotinate, acepimox, niceritrol, nicoclonate, nicomol and oxiniacic acid.
e. Thyroid Hormones and Analogs

Non-limiting examples of thyroid hormones and analogs thereof include etoroxate, thyropropic acid and thyroxine.
f. Miscellaneous Antihyperlipoproteinemics

Non-limiting examples of miscellaneous antihyperlipoproteinemics include acifran, azacosterol, benfluorex, b-benzalbutyramide, carnitine, chondroitin sulfate, clomestrone, detaxtran, dextran sulfate sodium, 5,8,11,14,17-eicosapentaenoic acid, eritadenine, furazabol, meglutol, melinamide, mytatrienediol, ornithine, g-oryzanol, pantethine, pentaerythritol tetraacetate, a-phenylbutyramide, pirozadil, probucol (lorelco), b-sitosterol, sultosilic acid-piperazine salt, tiadenol, triparanol and xenbucin.
2. Antiarteriosclerotics

Non-limiting examples of an antiarteriosclerotic include pyridinol carbamate.
3. Antithrombotic/Fibrinolytic Agents

In certain embodiments, administration of an agent that aids in the removal or prevention of blood clots may be combined with administration of a modulator, particularly in treatment of athersclerosis and vasculature (e.g., arterial) blockages. Non-limiting examples of antithrombotic and/or fibrinolytic agents include anticoagulants, anticoagulant antagonists, antiplatelet agents, thrombolytic agents, thrombolytic agent antagonists or combinations thereof.

In certain aspects, antithrombotic agents that can be administered orally, such as, for example, aspirin and wafarin (coumadin), are preferred.
a. Anticoagulants

A non-limiting example of an anticoagulant include acenocoumarol, ancrod, anisindione, bromindione, clorindione, coumetarol, cyclocumarol, dextran sulfate sodium, dicumarol, diphenadione, ethyl biscoumacetate, ethylidene dicoumarol, fluindione, heparin, hirudin, lyapolate sodium, oxazidione, pentosan polysulfate, phenindione, phenprocoumon, phosvitin, picotamide, tioclomarol and warfarin.
b. Antiplatelet Agents

Non-limiting examples of antiplatelet agents include aspirin, a dextran, dipyridamole (persantin), heparin, sulfinpyranone (anturane) and ticlopidine (ticlid).
c. Thrombolytic Agents

Non-limiting examples of thrombolytic agents include tissue plasminogen activator (activase), plasmin, pro-urokinase, urokinase (abbokinase) streptokinase (streptase), anistreplase/APSAC (eminase).
4. Blood Coagulants

In certain embodiments wherein a patient is suffering from a hemorrhage or an increased likelihood of hemorrhaging, an agent that may enhance blood coagulation may be used. Non-limiting examples of a blood coagulation promoting agent include thrombolytic agent antagonists and anticoagulant antagonists.
a. Anticoagulant Antagonists

Non-limiting examples of anticoagulant antagonists include protamine and vitamine K1.
b. Thrombolytic Agent Antagonists and Antithrombotics

Non-limiting examples of thrombolytic agent antagonists include aminocaproic acid (amicar) and tranexamic acid (amstat). Non-limiting examples of antithrombotics include anagrelide, argatroban, cilstazol, daltroban, defibrotide, enoxaparin, fraxiparine, indobufen, lamoparan, ozagrel, picotamide, plafibride, tedelparin, ticlopidine and triflusal.
5. Antiarrhythmic Agents

Non-limiting examples of antiarrhythmic agents include Class I antiarrhythmic agents (sodium channel blockers), Class II antiarrhythmic agents (beta-adrenergic blockers), Class II antiarrhythmic agents (repolarization prolonging drugs), Class IV antiarrhythmic agents (calcium channel blockers) and miscellaneous antiarrhythmic agents.
a. Sodium Channel Blockers

Non-limiting examples of sodium channel blockers include Class IA, Class IB and Class IC antiarrhythmic agents. Non-limiting examples of Class IA antiarrhythmic agents include disppyramide (norpace), procainamide (pronestyl) and quinidine (quinidex). Non-limiting examples of Class IB antiarrhythmic agents include lidocaine (xylocalne), tocamide (tonocard) and mexiletine (mexitil). Non-limiting examples of Class IC antiarrhythmic agents include encamide (enkaid) and flecamide (tambocor).
b. Beta Blockers

Non-limiting examples of a beta blocker, otherwise known as a b-adrenergic blocker, a b-adrenergic antagonist or a Class II antiarrhythmic agent, include acebutolol (sectral), alprenolol, amosulalol, arotinolol, atenolol, befimolol, betaxolol, bevantolol, bisoprolol, bopindolol, bucumolol, bufetolol, bufuralol, bunitrolol, bupranolol, butidrine hydrochloride, butofilolol, carazolol, carteolol, carvedilol, celiprolol, cetamolol, cloranolol, dilevalol, epanolol, esmolol (brevibloc), indenolol, labetalol, levobunolol, mepindolol, metipranolol, metoprolol, moprolol, nadolol, nadoxolol, nifenalol, nipradilol, oxprenolol, penbutolol, pindolol, practolol, pronethalol, propanolol (inderal), sotalol (betapace), sulfinalol, talinolol, tertatolol, timolol, toliprolol and xibinolol. In certain aspects, the beta blocker comprises an aryloxypropanolamine derivative. Non-limiting examples of aryloxypropanolamine derivatives include acebutolol, alprenolol, arotinolol, atenolol, betaxolol, bevantolol, bisoprolol, bopindolol, bunitrolol, butofilolol, carazolol, carteolol, carvedilol, celiprolol, cetamolol, epanolol, indenolol, mepindolol, metipranolol, metoprolol, moprolol, nadolol, nipradilol, oxprenolol, penbutolol, pindolol, propanolol, talinolol, tertatolol, timolol and toliprolol.
c. Repolarization Prolonging Agents

Non-limiting examples of an agent that prolong repolarization, also known as a Class III antiarrhythmic agent, include amiodarone (cordarone) and sotalol (betapace).
d. Calcium Channel Blockers/Antagonist

Non-limiting examples of a calcium channel blocker, otherwise known as a Class IV antiarrhythmic agent, include an arylalkylamine (e.g., bepridile, diltiazem, fendiline, gallopamil, prenylamine, terodiline, verapamil), a dihydropyridine derivative (felodipine, isradipine, nicardipine, nifedipine, nimodipine, nisoldipine, nitrendipine) a piperazinde derivative (e.g., cinnarizine, flunarizine, lidoflazine) or a miscellaneous calcium channel blocker such as bencyclane, etafenone, magnesium, mibefradil or perhexyline. In certain embodiments a calcium channel blocker comprises a long-acting dihydropyridine (amlodipine) calcium antagonist.
e. Miscellaneous Antiarrhythmic Agents

Non-limiting examples of miscellaneous antiarrhymic agents include adenosine (adenocard), digoxin (lanoxin), acecainide, ajmaline, amoproxan, aprindine, bretylium tosylate, bunaftine, butobendine, capobenic acid, cifenline, disopyranide, hydroquinidine, indecamide, ipatropium bromide, lidocaine, lorajmine, lorcamide, meobentine, moricizine, pirmenol, prajmaline, propafenone, pyrinoline, quinidine polygalacturonate, quinidine sulfate and viquidil.
6. Antihypertensive Agents

Non-limiting examples of antihypertensive agents include sympatholytic, alpha/beta blockers, alpha blockers, anti-angiotensin II agents, beta blockers, calcium channel blockers, vasodilators and miscellaneous antihypertensives.
a. Alpha Blockers

Non-limiting examples of an alpha blocker, also known as an a-adrenergic blocker or an a-adrenergic antagonist, include amosulalol, arotinolol, dapiprazole, doxazocin, ergoloid mesylates, fenspiride, indoramin, labetalol, nicergoline, prazosin, terazosin, tolazoline, trimazosin and yohimbine. In certain embodiments, an alpha blocker may comprise a quinazoline derivative. Non-limiting examples of quinazoline derivatives include alfuzosin, bunazosine, doxazocin, prazosin, terazosin and trimazosin.
b. Alpha/Beta Blockers

In certain embodiments, an antihypertensive agent is both an alpha and beta adrenergic antagonist. Non-limiting examples of an alpha/beta blocker comprise labetalol (normodyne, trandate).
c. Anti-Angiotension II Agents

Non-limiting examples of anti-angiotension II agents include angiotensin converting enzyme inhibitors and angiotension II receptor antagonists. Non-limiting examples of angiotension converting enzyme inhibitors (ACE inhibitors) include alacepril, enalapril (vasotec), captopril, cilazapril, delapril, enalaprilat, fosinopril, lisinopril, moveltopril, perindopril, quinapril and ramipril. Non-limiting examples of an angiotensin II receptor blocker, also known as an angiotension II receptor antagonist, an ANG receptor blocker or an ANG-II type-i receptor blocker (ARBS), include angiocandesartan, eprosartan, irbesartan, losartan and valsartan.
d. Sympatholytics

Non-limiting examples of a sympatholytic include a centrally acting sympatholytic or a peripherally acting sympatholytic. Non-limiting examples of a centrally acting sympatholytic, also known as an central nervous system (CNS) sympatholytic, include clonidine (catapres), guanabenz (wytensin) guanfacine (tenex) and methyldopa (aldomet). Non-limiting examples of a peripherally acting sympatholytic include a ganglion blocking agent, an adrenergic neuron blocking agent, a β-adrenergic blocking agent or a alpha1-adrenergic blocking agent. Non-limiting examples of a ganglion blocking agent include mecamylamine (inversine) and trimethaphan (arfonad). Non-limiting of an adrenergic neuron blocking agent include guanethidine (ismelin) and reserpine (serpasil). Non-limiting examples of a β-adrenergic blocker include acenitolol (sectral), atenolol (tenormin), betaxolol (kerlone), carteolol (cartrol), labetalol (normodyne, trandate), metoprolol (lopressor), nadanol (corgard), penbutolol (levatol), pindolol (visken), propranolol (inderal) and timolol (blocadren). Non-limiting examples of alpha1-adrenergic blocker include prazosin (minipress), doxazocin (cardura) and terazosin (hytrin).
e. Vasodilators

In certain embodiments a cardiovasculator therapeutic agent may comprise a vasodilator (e.g., a cerebral vasodilator, a coronary vasodilator or a peripheral vasodilator). In certain preferred embodiments, a vasodilator comprises a coronary vasodilator. Non-limiting examples of a coronary vasodilator include amotriphene, bendazol, benfurodil hemisuccinate, benziodarone, chloracizine, chromonar, clobenfurol, clonitrate, dilazep, dipyridamole, droprenilamine, efloxate, erythrityl tetranitrane, etafenone, fendiline, floredil, ganglefene, herestrol bis(b-diethylaminoethyl ether), hexobendine, itramin tosylate, khellin, lidoflanine, mannitol hexanitrane, medibazine, nicorglycerin, pentaerythritol tetranitrate, pentrinitrol, perhexyline, pimethylline, trapidil, tricromyl, trimetazidine, troInitrate phosphate and visnadine.

In certain aspects, a vasodilator may comprise a chronic therapy vasodilator or a hypertensive emergency vasodilator. Non-limiting examples of a chronic therapy vasodilator include hydralazine (apresoline) and minoxidil (loniten). Non-limiting examples of a hypertensive emergency vasodilator include nitroprusside (nipride), diazoxide (hyperstat IV), hydralazine (apresoline), minoxidil (loniten) and verapamil.
f. Miscellaneous Antihypertensives

Non-limiting examples of miscellaneous antihypertensives include ajmaline, g aminobutyric acid, bufeniode, cicletainine, ciclosidomine, a cryptenamine tannate, fenoldopam, flosequinan, ketanserin, mebutamate, mecamylamine, methyldopa, methyl 4-pyridyl ketone thiosemicarbazone, muzolimine, pargyline, pempidine, pinacidil, piperoxan, primaperone, a protoveratrine, raubasine, rescimetol, rilmenidene, saralasin, sodium nitrorusside, ticrynafen, trimethaphan camsylate, tyrosinase and urapidil.

In certain aspects, an antihypertensive may comprise an arylethanolamine derivative, a benzothiadiazine derivative, a N-carboxyalkyl(peptide/lactam) derivative, a dihydropyridine derivative, a guanidine derivative, a hydrazines/phthalazine, an imidazole derivative, a quanternary ammonium compound, a reserpine derivative or a suflonamide derivative.

Arylethanolamine Derivatives. Non-limiting examples of arylethanolamine derivatives include amosulalol, bufuralol, dilevalol, labetalol, pronethalol, sotalol and sulfinalol.

Benzothiadiazine Derivatives. Non-limiting examples of benzothiadiazine derivatives include althizide, bendroflumethiazide, benzthiazide, benzylhydrochlorothiazide, buthiazide, chlorothiazide, chlorthalidone, cyclopenthiazide, cyclothiazide, diazoxide, epithiazide, ethiazide, fenquizone, hydrochlorothizide, hydroflumethizide, methyclothiazide, meticrane, metolazone, paraflutizide, polythizide, tetrachlormethiazide and trichlormethiazide.

N-carboxyalkyl(peptide/lactam) Derivatives. Non-limiting examples of N-carboxyalkyl(peptide/lactam) derivatives include alacepril, captopril, cilazapril, delapril, enalapril, enalaprilat, fosinopril, lisinopril, moveltipril, perindopril, quinapril and ramipril.

Dihydropyridine Derivatives. Non-limiting examples of dihydropyridine derivatives include amlodipine, felodipine, isradipine, nicardipine, nifedipine, nilvadipine, nisoldipine and nitrendipine.

Guanidine Derivatives. Non-limiting examples of guanidine derivatives include bethanidine, debrisoquin, guanabenz, guanacline, guanadrel, guanazodine, guanethidine, guanfacine, guanochlor, guanoxabenz and guanoxan.

Hydrazines/Phthalazines. Non-limiting examples of hydrazines/phthalazines include budralazine, cadralazine, dihydralazine, endralazine, hydracarbazine, hydralazine, pheniprazine, pildralazine and todralazine.

Imidazole Derivatives. Non-limiting examples of imidazole derivatives include clonidine, lofexidine, phentolamine, tiamenidine and tolonidine.

Quanternary Ammonium Compounds. Non-limiting examples of quanternary ammonium compounds include azamethonium bromide, chlorisondamine chloride, hexamethonium, pentacynium bis(methylsulfate), pentamethonium bromide, pentolinium tartrate, phenactropinium chloride and trimethidinium methosulfate.

Reserpine Derivatives. Non-limiting examples of reserpine derivatives include bietaserpine, deserpidine, rescinnamine, reserpine and syrosingopine.

Suflonamide Derivatives. Non-limiting examples of sulfonamide derivatives include ambuside, clopamide, furosemide, indapamide, quinethazone, tripamide and xipamide.
7. Vasopressors

Vasopressors generally are used to increase blood pressure during shock, which may occur during a surgical procedure. Non-limiting examples of a vasopressor, also known as an antihypotensive, include amezinium methyl sulfate, angiotensin amide, dimetofrine, dopamine, etifelmin, etilefrin, gepefrine, metaraminol, midodrine, norepinephrine, pholedrine and synephrine.
8. Treatment Agents for Congestive Heart Failure

Non-limiting examples of agents for the treatment of congestive heart failure include anti-angiotension II agents, afterload-preload reduction treatment, diuretics and inotropic agents.
a. Afterload-Preload Reduction

In certain embodiments, an animal patient that can not tolerate an angiotension antagonist may be treated with a combination therapy. Such therapy may combine administration of hydralazine (apresoline) and isosorbide dinitrate (isordil, sorbitrate).
b. Diuretics

Non-limiting examples of a diuretic include a thiazide or benzothiadiazine derivative (e.g., althiazide, bendroflumethazide, benzthiazide, benzylhydrochlorothiazide, buthiazide, chlorothiazide, chlorothiazide, chlorthalidone, cyclopenthiazide, epithiazide, ethiazide, ethiazide, fenquizone, hydrochlorothiazide, hydroflumethiazide, methyclothiazide, meticrane, metolazone, paraflutizide, polythizide, tetrachloromethiazide, trichlormethiazide), an organomercurial (e.g., chlormerodrin, meralluride, mercamphamide, mercaptomerin sodium, mercumallylic acid, mercumatilin dodium, mercurous chloride, mersalyl), a pteridine (e.g., furterene, triamterene), purines (e.g., acefylline, 7-morpholinomethyltheophylline, pamobrom, protheobromine, theobromine), steroids including aldosterone antagonists (e.g., canrenone, oleandrin, spironolactone), a sulfonamide derivative (e.g., acetazolamide, ambuside, azosemide, bumetanide, butazolamide, chloraminophenamide, clofenamide, clopamide, clorexolone, diphenylmethane-4,4′-disulfonamide, disulfamide, ethoxzolamide, furosemide, indapamide, mefruside, methazolamide, piretanide, quinethazone, torasemide, tripamide, xipamide), a uracil (e.g., aminometradine, amisometradine), a potassium sparing antagonist (e.g., amiloride, triamterene) or a miscellaneous diuretic such as aminozine, arbutin, chlorazanil, ethacrynic acid, etozolin, hydracarbazine, isosorbide, mannitol, metochalcone, muzolimine, perhexyline, ticmafen and urea.
c. Inotropic Agents

Non-limiting examples of a positive inotropic agent, also known as a cardiotonic, include acefylline, an acetyldigitoxin, 2-amino-4-picoline, aminone, benfurodil hemisuccinate, bucladesine, cerberosine, camphotamide, convallatoxin, cymarin, denopamine, deslanoside, digitalin, digitalis, digitoxin, digoxin, dobutamine, dopamine, dopexamine, enoximone, erythrophleine, fenalcomine, gitalin, gitoxin, glycocyamine, heptaminol, hydrastinine, ibopamine, a lanatoside, metamivam, milrinone, nerifolin, oleandrin, ouabain, oxyfedrine, prenalterol, proscillaridine, resibufogenin, scillaren, scillarenin, strphanthin, sulmazole, theobromine and xamoterol.

In particular aspects, an intropic agent is a cardiac glycoside, a beta-adrenergic agonist or a phosphodiesterase inhibitor. Non-limiting examples of a cardiac glycoside includes digoxin (lanoxin) and digitoxin (crystodigin). Non-limiting examples of a β-adrenergic agonist include albuterol, bambuterol, bitolterol, carbuterol, clenbuterol, clorprenaline, denopamine, dioxethedrine, dobutamine (dobutrex), dopamine (intropin), dopexamine, ephedrine, etafedrine, ethylnorepinephrine, fenoterol, formoterol, hexoprenaline, ibopamine, isoetharine, isoproterenol, mabuterol, metaproterenol, methoxyphenamine, oxyfedrine, pirbuterol, procaterol, protokylol, reproterol, rimiterol, ritodrine, soterenol, terbutaline, tretoquinol, tulobuterol and xamoterol. Non-limiting examples of a phosphodiesterase inhibitor include aminone (inocor).
d. Antianginal Agents

Antianginal agents may comprise organonitrates, calcium channel blockers, beta blockers and combinations thereof. Non-limiting examples of organonitrates, also known as nitrovasodilators, include nitroglycerin (nitro-bid, nitrostat), isosorbide dinitrate (isordil, sorbitrate) and amyl nitrate (aspirol, vaporole).
9. Surgical Therapeutic Agents

In certain aspects, the secondary therapeutic agent may comprise a surgery of some type, which includes, for example, preventative, diagnostic or staging, curative and palliative surgery. Surgery, and in particular a curative surgery, may be used in conjunction with other therapies, such as the present invention and one or more other agents.

Such surgical therapeutic agents for vascular and cardiovascular diseases and disorders are well known to those of skill in the art, and may comprise, but are not limited to, performing surgery on an organism, providing a cardiovascular mechanical prostheses, angioplasty, coronary artery reperfusion, catheter ablation, providing an implantable cardioverter defibrillator to the subject, mechanical circulatory support or a combination thereof. Non-limiting examples of a mechanical circulatory support that may be used in the present invention comprise an intra-aortic balloon counterpulsation, left ventricular assist device or combination thereof.

E. Drug Formulations and Routes for Administration to Patients

It will be understood that in the discussion of formulations and methods of treatment, references to any compounds are meant to also include the pharmaceutically acceptable salts, as well as pharmaceutical compositions. Where clinical applications are contemplated, pharmaceutical compositions will be prepared in a form appropriate for the intended application. Generally, this will entail preparing compositions that are essentially free of pyrogens, as well as other impurities that could be harmful to humans or animals.

One will generally desire to employ appropriate salts and buffers to render delivery vectors stable and allow for uptake by target cells. Buffers also will be employed when recombinant cells are introduced into a patient. Aqueous compositions of the present invention comprise an effective amount of the vector or cells, dissolved or dispersed in a pharmaceutically acceptable carrier or aqueous medium. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes solvents, buffers, solutions, dispersion media, coatings, antibacterial and antifungal agents, isotonic and absorption delaying agents and the like acceptable for use in formulating pharmaceuticals, such as pharmaceuticals suitable for administration to humans. The use of such media and agents for pharmaceutically active substances is well known in the art. Except insofar as any conventional media or agent is incompatible with the active ingredients of the present invention, its use in therapeutic compositions is contemplated. Supplementary active ingredients also can be incorporated into the compositions, provided they do not inactivate the vectors or cells of the compositions.

In specific embodiments of the invention the pharmaceutical formulation will be formulated for delivery via rapid release, other embodiments contemplated include but are not limited to timed release, delayed release, and sustained release. Formulations can be an oral suspension in either the solid or liquid form. In further embodiments, it is contemplated that the formulation can be prepared for delivery via parenteral delivery, or used as a suppository, or be formulated for subcutaneous, intravenous, intramuscular, intraperitoneal, sublingual, transdermal, or nasopharyngeal delivery.

The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients, which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example, magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the technique described in the U.S. Pat. Nos. 4,256,108; 4,166,452; and 4,265,874 to form osmotic therapeutic tablets for control release (hereinafter incorporated by reference).

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil.

Aqueous suspensions contain an active material in admixture with excipients suitable for the manufacture of aqueous suspensions. Such excipients are suspending agents, for example sodium carboxymethylcellulose, methylcellulose, hydroxy-propylmethycellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

Pharmaceutical compositions may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavouring agents.

Syrups and elixirs may be formulated with sweetening agents, for example glycerol, propylene glycol, sorbitol or sucrose. Such formulations may also contain a demulcent, a preservative and flavoring and coloring agents. Pharmaceutical compositions may be in the form of a sterile injectable aqueous or oleagenous suspension. Suspensions may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Compounds may also be administered in the form of suppositories for rectal administration of the drug. These compositions can be prepared by mixing a therapeutic agent with a suitable non-irritating excipient which is solid at ordinary temperatures, but liquid at the rectal temperature and will therefore melt in the rectum to release the drug. Such materials are cocoa butter and polyethylene glycols. For topical use, creams, ointments, jellies, gels, epidermal solutions or suspensions, etc., containing a therapeutic compound are employed. For purposes of this application, topical application shall include mouthwashes and gargles. Formulations may also be administered as nanoparticles, liposomes, granules, inhalants, nasal solutions, or intravenous admixtures

The amount of active ingredient in any formulation may vary to produce a dosage form that will depend on the particular treatment and mode of administration. It is further understood that specific dosing for a patient will depend upon a variety of factors including age, body weight, general health, sex, diet, time of administration, route of administration, rate of excretion, drug combination and the severity of the particular disease undergoing therapy.

### V. EXAMPLES

The following examples are included to further illustrate various aspects of the invention. It should be appreciated by those of skill in the art that the techniques disclosed in the examples which follow represent techniques and/or compositions discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

Reagents. N⁶-cyclopentyladenosine (CPA), 5′-N-ethylcarboxamidoadenosine (NECA), 4-((N-ethyl-5′-carbamoyladenos-2-yl)-aminoethyl)-phenyl-propionic acid (CGS21680), 8-cyclopentyl-1,3-dipropylxanthine (DPCPX), and adenosine were purchased from Sigma (St. Louis, Mo.). Endonorbornan-2-yl-9-methyladenine (N-0861) was a gift from Whitby Research, Inc. (Richmond, Va.) and 5-amino-7-(phenylethyl)-2-(2-furyl)-pyrazolo-[4,3-e]-1,2,4-triazolo-[1,5-c]-pyrimidine (SCH58261) was a gift from Drs C. Zocchi and E. Ongini (Schering Plough Research Institute, Milan, Italy). 3-isobutyl-8-pyrrolidinoxanthine (IPDX) was synthesized as previously described (Feoktisov et al., 2001). Dimethyl sulfoxide (DMSO) was purchased from Sigma. When used as a solvent, final DMSO concentrations in all assays did not exceed 1% and the same DMSO concentrations were used in vehicle controls.

Cell isolation and culture. MCEC-1, conditionally immortalized mouse cardiac microvascular endothelial cells, were generously provided by Dr. J. Mason (National Heart and Lung Institute, UK). The cells were isolated from H-2 K^{b}-tsA58 transgenic mice containing a gene encoding the thermolabile SV40 T antigen. Cell cultures were propagated in the presence of 20 U/mL recombinant mouse IFN-γ (PeproTech, Rocky Hill, N.J.) at 33° C. on 1% gelatin-coated tissue culture plates containing DMEM supplemented with 10% FBS, Antibiotic-Antimycotic mixture (Sigma), 2 mmol/L L-glutamine, 10 U/mL heparin, and 30 μg/mL ECGF. Six days before experiments, cells were replated and cultured in the absence of IFN-γ at 37° C. Under these conditions, MCEC-1 cells assume the phenotype of primary cardiac microvascular endothelial cells (Lidington et al., 2002).

Primary cultures of human cardiac microvascular endothelial cells (HMVEC-c) were obtained from Cambrex (Walkersville, Md.), and cultured using EGM™-2 MV growth medium (Cambrex). HMVEC-c from passages 2 to 5 were used.

Mouse endothelial progenitor cells isolated from E7.5 embryos (eEPCs) have been previously described (Hatzopoulos et al., 1998). Cells were maintained in DMEM medium supplemented with 20% FBS, 2 mmol/L L-glutamine, 1 mmol/L pyruvic acid, MEM nonessential Amino Acids (Mediatech Inc, Hemdon, Va.), Antibiotic Antimycotic mixture (Sigma) and 0.1 mmol/L β-mercaptoethanol.

Normal human peripheral blood leukocytes were obtained from human blood donor leukocyte reduction filters (LeukotrapRC, Pall Corporation, East Hills, N.Y.) otherwise discarded by the American Red Cross (Nashville, Tenn.) as previously described (Teleron et al., 2005); three to four filters were pooled per prep to reduce donor variability. Mononuclear cells from leukocytes were obtained by centrifugation on Histopaque 1077 (Sigma) gradients according to manufacturer instructions. Mononuclear cells were directly plated at 10⁸ cells/cm² culture dishes and maintained in EBM-2 (Clonetics) with supplements according to previously published protocol (Teleron et al., 2005). EPCs were harvested on day 7 and were identified by uptake of DiI-acLDL and co-staining with UEA-1 lectin as well as anti-VEGFR2 and VE-cadherin by indirect immunofluorescence as described previously (Teleron et al., 2005).

Human CD34⁺ cells were purified from normal peripheral blood mononuclear cells using the direct CD34⁺ progenitor cell MACS isolation kit (Miltenyi Biotec, Gladbach, Germany), according to the manufacturer's recommendations. Flow cytometry using a monoclonal CD34-PE antibody (clone AC136) and CD45-FITC (Miltenyi Biotec) demonstrated 95% purity of isolated cells.

Measurement of cAMP accumulation. Cyclic AMP accumulation was measured as previously described (Feoktisov et al., 2002). Cells growing in 12-well plates were pre-incubated in 150 mmol/L NaCl, 2.7 mmol/L KCl, 0.37 mmol/L NaH₂PO₄, 1 mmol/L MgSO₄, 1 mmol/L CaCl₂, 5 g/L D-glucose, 10 mmol/L HEPES-NaOH, pH 7.4 and 1 U/mL adenosine deaminase containing the cAMP phosphodiesterase inhibitor papaverine (1 mmol/L) for 15 min at 37° C. Adenosine agonists and antagonists were added to cells, and the incubation was allowed to proceed for 3 min at 37° C. The reaction was stopped by the addition of ⅕ volume of 25% trichloroacetic acid. The extracts were washed five times with 10 volumes of water-saturated ether. Cyclic AMP concentrations were determined using a cAMP assay kit (GE Healthcare, Little Chalfont, UK).

Real-time reverse transcription-polymerase chain reaction (RT-PCR). Real-time RT-PCR analysis was performed as previously described (Ryzhov et al., 2007). Total RNA was isolated from cells using RNeasy Mini kit (Qiagen, Valencia, Calif.). Real-time RT-PCR was carried out on ABI PRISM 7900HT Sequence Detection System (PE Applied Biosystems, Foster City, Calif.). Primer pairs and FAM-labeled probes for murine and human adenosine receptors and β-actin were provided by Applied Biosystems. RT-PCR reactions utilizing 1 μg of DNase-treated total RNA were performed under conditions recommended by the manufacturer. A standard curve for each amplicon was obtained using serial dilutions of total RNA. The results from triplicate polymerase chain reactions for a given gene at each time point were used to determine mRNA quantity relative to the corresponding standard curve. The relative mRNA quantity for a given gene measured from a single reverse transcription reaction was divided by the value obtained for β-actin to correct for fluctuations in input RNA levels and varying efficiencies of reverse transcription reactions.

Analysis of cell adhesion under static conditions. Endothelial cells were grown to confluency in 96-well plates. One hour before experiments, the growth medium in each well was replaced with 70 μl of DMEM. Progenitor cells were fluorescently labeled by incubating with 5 μmol/L calcein-AM (Molecular Probes, Eugene, Oreg.) in DMEM (10⁷ cells/mL) for 30 min at 37° C. Labeled cells were washed three times by centrifugation and resuspended in DMEM (10⁶ cells/mL). In some experiments, eEPCs were pre-incubated with 10 μg/mL rat monoclonal anti-mouse PSGL-1 antibody (clone 2PH1, Fitzgerald Industries, Concord, Mass.) or control rat IgG, (BD Biosciences, San Jose, Calif.) for 15 min at room temperature. The assay was started by transferring 50 μl of labeled cell suspension to each well covered with endothelial monolayer followed immediately by addition of 30 μl of DMEM containing 5× concentrations of test compounds or controls. Plates were placed in a cell culture incubator at 37° C. At the end of incubation periods indicated in the Results section, below, 96-well plates were gently washed twice with DMEM and twice with Tyrode's buffer (150 mmol/L NaCl, 2.7 mmol/L KCl, 0.37 mmol/L NaH₂PO₄, 1 mmol/L MgSO₄, 1 mmol/L CaCl₂, 5 g/L D-glucose, 10 mmol/L HEPES-NaOH, pH 7.4) and finally 150 μl of Tyrode's buffer was added to each well. Cell adhesion was measured using a fluorescence plate reader at excitation and emission wavelengths of 485 and 535 nm, respectively. The percentage of adhered fluorescent cells was calculated using a calibration curve constructed for each experiment by measuring fluorescence of predetermined numbers of labeled cells. Results section, 96-well plates were gently washed twice with DMEM and twice with Tyrode's buffer (150 mmol/L NaCl, 2.7 mmol/L KCl, 0.37 mmol/L NaH₂PO₄, 1 mmol/L MgSO₄.

Analysis of cell adhesion under flow conditions. Adhesion assays under flow conditions were performed using a parallel plate flow chamber (Glycotech, Rockville, Md.) following the manufacturer's instructions. Cell suspension or cell-free medium were drown into chambers by a syringe pump (Model 44, Harvard Apparatus, Inc., Holliston, Mass.) at a constant rate to generate a desired wall shear stress (τ, dynes/cm₂) using the formula τ=6Qμ/a²b, where Q is flow rate, μ is medium viscosity, b is channel width, and a is channel height. After flow chamber assembly, the endothelial monolayer was perfused for 10 min with DMEM containing 10 μmol/L NECA or its vehicle, and then with an EPC suspension in the same medium for another 10 min. Cells were observed with a Nikon model TMS inverted phase contrast microscope (Nikon USA, Melville, N.Y.) and videotaped with a Sony DCR-TRV480 color video camera (Sony Corporation, Tokyo, Japan). Cell adhesion was determined by analysis of digitized video recordings using NIH Image software. Cell-based P-selectin enzyme-linked immunoassay To analyze cell-surface P-selectin expression, the inventors used a previously published method (Cleator et al., 2005). In brief, MCEC-1 cells were incubated in the presence of 10 μmol/L NECA or its vehicle (DMSO) at 37° C. for periods indicated and then fixed for 5 min with 0.5% paraformaldehyde solution. After washing and blocking, cells were incubated with 5 μg/mL rat anti-mouse CD62P antibodies (Fitzgerald Industries) or rat isotype-matched control antibody (BD Biosciences) for 1 hour. After washing, a secondary goat anti-rat horseradish peroxidase-conjugated antibody (Jackson ImmunoResearch, West Grove, Pa.) was added for 1 hour followed by washing and then analyzed at 450 nM after addition of substrate.

Isolated mouse heart model. Six male C57Bl/6 mice (Jackson Laboratory, Bar Harbor, Me.) at age of 6-8 weeks were used. The study was conducted in accordance with the Guide for the Care and Use of Laboratory Animals as adopted and promulgated by the U.S. National Institutes of Health. Hearts were rapidly removed from mice anesthetized with inhalation of isoflurane. The aorta was cannulated and connected to a Langendorff apparatus. The Langendorff perfusion was carried out at a constant flow rate of 4 mL/min with modified Krebs-Henseleit (KH) buffer (118 mmol/L NaCl, 25 mmol/L NaHCO₃, 4.7 mmol/L KCl, 1.2 mmol/L MgSO₄, 1.2 mmol/L NaH₂PO₄, 2.5 mmol/L CaCl₂, 11 mmol/L glucose, 0.5 mmol/L EDTA, pH 7.4) equilibrated with a gas mixture of 95% O₂ and 5% CO₂ at 37° C. After a 30 min stabilization period, hearts were perfused with 1.5 mmol/L FITC-conjugated Helix pomatia lectin (Sigma) for 10 min to label endothelial cells of perfused vessels followed by a 10 min washing period with KH buffer. Hearts were then perfused with eEPCs pre-labeled with DiI C 16 (Invitrogen, Carlsbad, Calif.) and resuspended in KH buffer containing 2% FBS (2,500 cells/mL) in the presence or absence of 10 μmol/L adenosine for 10 min. After washing with KH buffer for 10 min to remove unbound eEPCs, hearts were dissected, and placed on a microscopic stage. Retention of eEPCs in hearts was analyzed by taking 10 random images of the left ventricle using epifluorescence microscopy (20× objective). Area of EPC-emitted fluorescence was measured using NIH ImageJ software and normalized to the area of vascular endothelium stained with FITC-lectin.

### Example 2

### Results

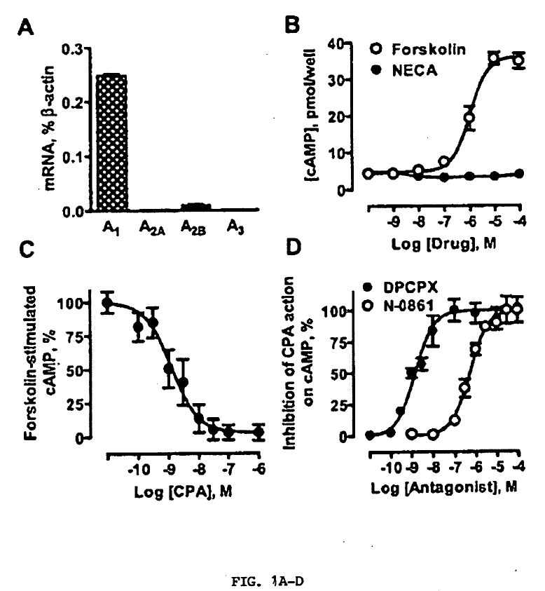

Adenosine Receptors in Mouse Embryonic EPCs. Real-time RT-PCR showed that eEPCs preferentially express mRNA encoding A1 receptors (0.248±0.004% of p-actin; FIG. 1A). Very low levels of A₂B receptor mRNA were also detected (0.009±0.002% of β-actin), whereas transcripts for A₂A and A₃ receptors were below detection levels.

The inventors measured cAMP accumulation as a way to determine whether expression of mRNA translates into functional presence of adenosine receptors in eEPCs; A₂A and A₂B receptors stimulate adenylate cyclase via coupling to Gs proteins, whereas A₁ and A₃ receptors inhibit this enzyme via coupling to Gi proteins.5 The affinity to adenosine receptor subtypes of the agonists and antagonists used are summarized in the Table 1.

TABLE 1
Affinity or potency of agonists and antagonists at human (h), rat (r), guinea pig
(gp) and mouse (m) adenosine receptor subtypes (Kᵢ, K_{D}, K_{B}, IC₅₀ or EC₅₀ values in
nmol/L with 95% confidence intervals or ±SEM in parentheses and in log mol/L).

| | Receptor subtypes | | |
|---|---|---|---|
| Compounds | A₁ | A_{2A} | A_{2B} |
| NECA | h 14 (6.4-29); −7.9 [1]* | h 20 (12-59); −7.7 [1] | h 330 (±60); −6.5 |
| | r 6.3 (±0.52); −8.2 [4] | r 10 (±0.5); −8 [4] | [2] |
| | r 11 (7-17); −8 [5] | r 22 (20-25); −7.7 [5] | h 360 (±120); −6.4 |
| | r 30 (21-43); −7.5 [6] | r 4.2 (3-5.9); −8.38 [6] | [3] |
| | m 15 (10-22); −7.8 [6] | | m 449 (291-693); −6.3† |
| CPA | h 2.3 (1.5-3.4); −8.6 [1] | h 790 (470-1,360); −6.1 | h 34,400 (±11,100); −4.5 |
| | r 0.59 (±0.02); −9.2 [4] | [1] | [2] |
| | r 0.8 (0.6-1.0); −9.1 [5] | r 460 (±15); −6.3 [4] | h 21,000(±4,300); −4.7 |
| | r 4 (2.8-5.8); −8.4 [6] | r 2,000 (1,400-2,900); −5.7 | [3] |
| | m 3.3 (0.9-12); −8.5 [6] | [5] | |
| | m 1.2 (0.6-2.4); −8.9 | r 148 (42-525); −6.8 [6] | |
| CGS21680 | h 290 (230-360); −6.5 | h 27 (12-59); −7.6 [1] | h 361,000 |
| | [1] | r 22 (±4.3); −7.7 [7] | (±21,000); −3.4 [2] |
| | r 3, 100 (±470); −5.5 [7] | r 3.6 (1.2-10.5); −8.44 | |
| | r 36,300 (20,000- | [6] | |
| | 66,100); −4.44 [6] | | |
| | m 14, 100 (7,000- | | |
| | 28,200); −4.85 [6] | | |
| DPCPX | h 3.9 (3.5-4.2); −8.4 [1] | h 129 (35-260); −6.9 [1] | h 50 (±3.7); −7.3 [2] |
| | r 0.3; −9.5 [8] | r 340; −6.5 [8] | h 51 (±6.1); −7.3 [3] |
| | r 2.8 (2.6-3.1); 9.55 [6] | r 151 (141-170); −6.8 | m 86 (±36); −7.1 [9] |
| | m 1.5 (±0.5); −8.8 [9] | [6] | |
| | m 0.4 (0.3-0.5); −9.3 [6] | m 598 (±71); −6.2 [9] | |
| | m 1.5 (1.1-2.1); −8.8 | | |
| SCH58261 | h 290 (210-410); −6.5 | h 0.6 (0.5-0.7); −9.2 | h > 100; >−5 [11] |
| | [10] | [10] | m 1,868 (1,404- |
| | r 120 (100-140); −6.9 | r 2.3 (2-2.7); −8.6 [12] | 2,486); −5.7 |
| | [12] | m 1.0 (0.4-1.6); −9.0 | |
| | m 854 (464-1,570); −6.1 | [13] | |
| IPDX | h 24,000 (±8,000); −4.6 | h 36,000 (±8,000); −4.4 | h 625 (±71); −6.2 |
| | [14] | [14] | [14] |
| | m 20,000 (16,230- | | m 603; −6.2 |
| | 24,870); −4.6 | | |
| N-0861 | | gp 575 (±86); 6.2 [15] | gp 56,200 |
| | | m 511 (398-656); −6.3 | (±11,400), −4.3 [15] |
| | | | m 39,350 (23,600- |
| | | | 65,610); −4.4 |

*data from references cited within brackets
†data from the current study are presented in boldface.
[1] Klotz et al. (1998); transfected CHO cells; radioligands [³H]CCPA (A₁), [³H]NECA (A_{2A}).
[2] Linden et al. (1999); transfected HEK 293 cells; radioligand ¹²⁵I-ABOPX.
[3] Ji and Jacobson (1999); transfected HEK 293 cells; radioligand [₃H]ZM241685.
[4] Bruns et al. (1986); brain membranes (A₁), striatum (A_{2A}); radioligands [³H]CHA (A₁), [³H]NECA(A_{2A}).
[5] Cristalli et al. (1992); brain membranes (A₁), striatum (A_{2A}); radioligands [³H]DPCPX (A₁), [³H]NECA (A_{2A}).
[6] Maemoto et al. (1997); brain cortex (A₁), striatum (A_{2A}); radioligands [³H]DPCPX (A₁), [³H]CGS21680 (A_{2A}).
[7] Hutchison et al. (1989); rat brain; radioligands [³H]CHA (A₁), [³H]NECA (A_{2A}).
[8] Lohse et al., 1987; brain membranes (A₁), striatum (A_{2A}); radioligands [³H]PIA (A₁), [₃H]NECA (A_{2A}).
[9] Kreckler et al. (2006); transfected HEK 293; radioligands [¹²⁵I]I-AB-MECA (A₁), [¹²⁵I]ZM241385 (A_{2A}), [³H]MRS1754 (A_{2B}).
[10] Ongini et al. (1999); transfected CHO cells (A₁ and A_{2A}) or HEK 293 cells (A_{2A}); radioligands [³H]DPCPX (A₁), [³H]SCH58261 (A_{2A}).
[11] Feoktistov and Biaggioni (1998); NECA-stimulated HEL cells.
[12] Baraldi et al. (1994); brain membranes (A₁), striatum (A_{2A}); radioligand [³H]CHA (A₁), [³H]CGS21680 (A_{2A}).
[13] Lopes et al. (2004); striatum, radioligand [³H]SCH58261.
[14] Feoktistov et al, 2001; transfected CHO cells (A₁ and A_{2A}), transfected HEK 293 or HEL cells (A_{2B}); radioligands [³H]DPCPX (A₁), [³H]NECA (A_{2A}), [³H]ZM241685 (A_{2B}).
[15] Martin et al. (1993); NECA-stimulated atrium (A₁) or aorta (A_{2A}).

Forskolin increased cAMP levels in eEPCs from 4.3±0.7 to 35±2 pmol per well, with an EC50 of 1.1 μmol/L, whereas the nonselective adenosine receptor agonist NECA did not elevate cAMP (FIG. 1B). This is contrary to what would be expected for activation of A₂B receptors. However, the selective A₁ agonist CPA inhibited forskolin-stimulated cAMP accumulation with an EC50 of 1.3 nmol/L (FIG. 1C), corresponding to its reported affinity at A₁ receptors (Fredholm et al., 2001). Furthermore, DPCPX and N-0861 antagonized the action of 10 nmol/L CPA on forskolin-stimulated cAMP accumulation with EC₅₀ values of 1.5 and 511 nmol/L, respectively (FIG. 1D), corresponding to their affinities at A1 receptors (Table 1). Thus, the inventors conclude that A₁ receptors are functionally present in eEPCs.

A₁ receptor transcripts were also detected (2.1±1.4% of β-actin) in human adult culture-expanded EPCs along with mRNA encoding other adenosine receptors (3.4±2.1%, 1.0±0.3%, and 0.3±0.1% of β-actin for A₂A, A₂B, and A₃ subtypes, respectively; n=4).

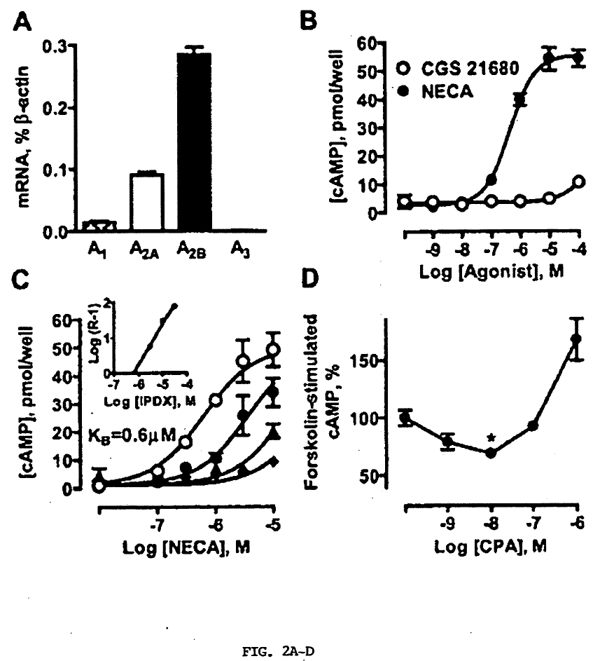

Adenosine Receptors in Cardiac Microvascular Endothelial Cells. Real-time RT-PCR analysis of MCEC-1 cells revealed preferential expression of mRNA encoding A₂B receptors (0.284±0.012% of β-actin), with lower expression of A₁ and A₂A receptors (0.016±0.002 and 0.091±0.005% of β-actin, respectively) and no detectable levels of A3 receptor transcripts (FIG. 2A).

NECA stimulated cAMP accumulation with an EC50 of 449 nmol/L, corresponding to its affinity at A2B receptors (Fredholm et al., 2001), whereas the A₂A agonist CGS21680 had no effect when used at selective concentrations (FIG. 2C). The selective A₂B antagonist IPDX progressively shifted concentration-response curves of NECA-stimulated cAMP accumulation to the right (FIG. 2C). Schild plot analysis (inset) determined that IPDX inhibits this A₂B-mediated process with a dissociation constant of 603 nmol/L, a value similar to that found in human cells (Feoktistov et al., 2001). Functional, albeit low, expression of A₁ receptors in MCEC-1 cells was also detected; the A₁ agonist CPA inhibited forskolin-stimulated adenylate cyclase at selective (low nanomolar) concentrations (Table 1). Inhibition was reversed with increasing concentrations of CPA (>100 nmol/L) presumably because of stimulation of A₂B receptors (FIG. 2D). Taken together, these data suggest that A₂B is the predominant receptor subtype regulating adenylate cyclase in MCEC-1 cells.

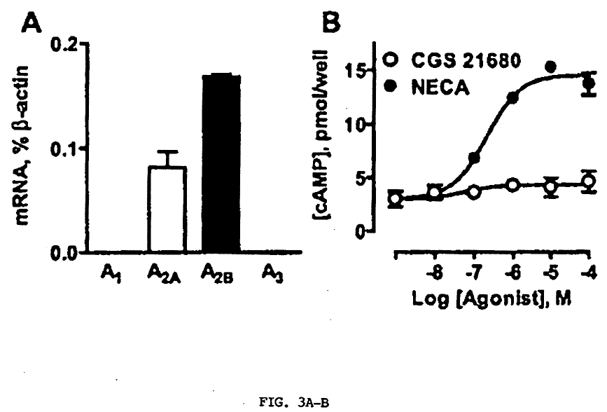

HMVEC-c cells preferentially expressed mRNA encoding A₂B receptors (0.168±0.003% of β-actin), lower levels of A₂A receptor transcripts (0.082±0.015% of β-actin), and no detectable levels of A₁ or A₃ receptor mRNA (FIG. 3A). Similarly to MCEC-1 cells, A2B receptor was the predominant subtype regulating adenylate cyclase in HMVEC-c cells; the non-selective agonist NECA stimulated cAMP accumulation 5.1±0.1-fold, whereas the selective A2A agonist CGS21680 had no significant effect (FIG. 3B).

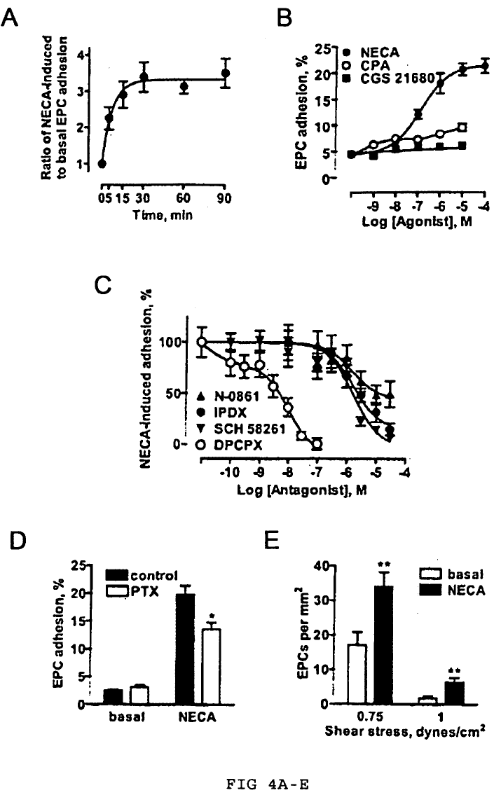

Role of Adenosine Receptors in EPC Adhesion to Cardiac Microvascular Endothelial Cells. Adhesion of fluorescently labeled mouse eEPCs to MCEC-1 cells was rapidly stimulated by 1 μmol/L NECA (FIGS. 4A and 4B), with a half-maximal effect observed at 5 minutes. Adhesion in the continuous presence of NECA was greater compared with adhesion in the absence of NECA after individual pretreatment of MCEC-1 cells and/or eEPCs with NECA. From data in FIG. 4A, the inventors selected 30 minutes as the incubation time that produced maximal increase in adhesion, and performed a pharmacological analysis of the adenosine receptor subtypes involved in this action. NECA increased eEPC adhesion from 4.5±0.3% to 21.3±1.4% in a concentration-dependent manner with an EC₅₀ of 139 nmol/L. Selective stimulation of A₁ receptors with 10 nmol/L CPA only slightly increased eEPC adhesion to 7.4±0.6%, whereas stimulation of A₂A receptors with CGS21680 had virtually no effect (FIG. 4C). Based on these results, the inventors selected 1 μmol/L NECA, a concentration producing submaximal increase in eEPC adhesion to MCEC-1 cells, to analyze the effects of adenosine receptorspecific antagonists. As seen in FIG. 4D, DPCPX, N-0861, and IPDX inhibited NECA-induced eEPC adhesion with IC₅₀ values of 4 nmol/L, 1.5 μmol/L, and 1.6 μmol/L, consistent with their respective potency at A₁ and A₂B receptors (Table 1). Of note, the selective A2A antagonist SCH58261 inhibited NECA-induced eEPC adhesion with an IC₅₀ value of 1.7 μmol/L that was consistent with its potency at A₁ and A₂B receptors, whereas it had no effect at lower concentrations that selectively block A₂A receptors (Table 1). Taken together, these data suggest that A₁ and A₂B, but not A₂A, receptors are involved in stimulation of eEPC adhesion to MCEC-1 cells by adenosine.

The inventors also used a complementary approach to evaluate the contribution of A1 receptors by preincubating eEPCs with 100 mmol/L pertussis toxin for 12 hours to uncouple the receptor to Gi proteins (Fredholm et al., 2001). In ancillary studies, the inventors documented that this treatment completely abrogated the ability of CPA to inhibit forskolin-stimulated adenylate cyclase, thus confirming the functional uncoupling of A1 receptors. Pertussis toxin treatment significantly attenuated but did not completely block the stimulation of adhesion induced by 10 μmol/L NECA (FIG. 4E). In contrast, this treatment had no effect on TNF-α-induced eEPC adhesion. Thus, the inventors conclude that stimulation of A₂B receptors on MCEC-1 cells is essential for EPC adhesion to endothelium, but that stimulation of A1 receptors on eEPCs can additionally increase their adherence. An increase in eEPC adhesion induced by stimulation of adenosine receptors can eventually lead to increased numbers of cells transmigrating endothelial layer, and the inventors studies indicate this possibility.

Next, the inventors evaluated the adhesion of these cells under laminar flow conditions by perfusing eEPCs over MCEC-1 monolayers at 2 different levels at the low end of physiologically relevant range of wall shear stress values (Jones et al., 1995), 0.75 and 1 dyne/cm² for 10 minutes. As expected, an increase in shear stress reduced adhesion of eEPCs to endothelial cells. However, stimulation of adenosine receptors with 10 μmol/L NECA significantly increased eEPC adhesion at both levels of shear stress (FIG. 4F). On stopping and resuming flow, the adhered eEPCs withstood further increase in laminar flow applied in increments of 1 dyne/cm² and started to detach only when shear stress exceeded 10 dyne/cm².

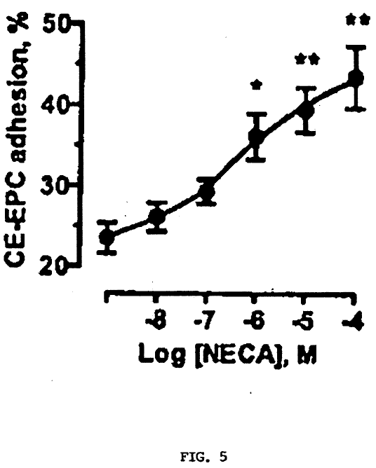

The inventors then measured the effect of adenosine receptor stimulation on the adhesion of human adult culture-expanded endothelial progenitor cells (CE-EPCs) to HMVEC-c cells. As seen in FIG. 5, NECA stimulated CE-EPC adhesion to HMVEC-c cells in a concentration-dependent manner. These results indicate that adenosine receptors can regulate not only adhesion of mouse embryonic EPCs but also homing of adult human progenitor cells to cardiac microvascular endothelial cells.

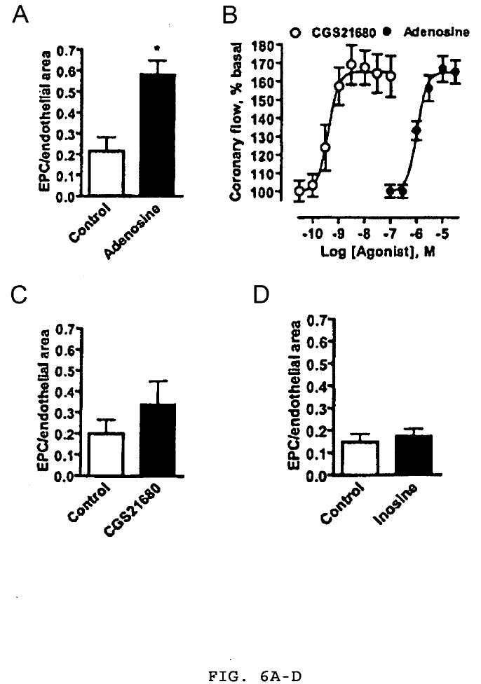

Adenosine Promotes EPC Retention in Isolated Mouse Hearts. To determine whether the observed adenosine-dependent increase in EPC adhesion to cardiac microvascular endothelial cells translates into increased retention of circulating EPCs in the coronary vasculature, the inventors used a conventional Langendorff retrograde perfusion system. Endothelial cells in coronary vessels were marked with FITC-conjugated Helix pomatia lectin (green; FIGS. 6A and 6B). Mouse eEPCs were labeled with DiI-C 16 to allow their detection at the surface of the left ventricle using epifluorescence microscopy (red; FIGS. 6C and 6D). FIGS. 6A-J shows representative images obtained from hearts perfused with eEPC suspension in the absence (FIGS. 6A, 6C and 6E) or presence of 10 μmol/L adenosine (FIGS. 6B, 6D and 6F). The inventors found that adenosine significantly increased the relative area of vascular network occupied by eEPCs (FIG. 6G).

A2A receptors are known to participate in adenosine-induced coronary vasodilation (Fredholm et al., 2001). Perfusion of hearts with the selective A2A agonist CGS21680 (3 nmol/L) produced comparable vasodilation as 10 μmol/L adenosine (FIG. 6H) but had a considerably less effect on eEPC retention (FIG. 6I), indicating that vasodilation per se cannot explain this phenomenon. In rodents, adenosine can also trigger the release of vasoactive compounds from mast cells via A3 receptors (Jin et al., 1997). However, stimulation of A3 receptors with 100 μmol/L inosine (Jin et al., 1997) had no effect on eEPC retention in perfused hearts (FIG. 6J).

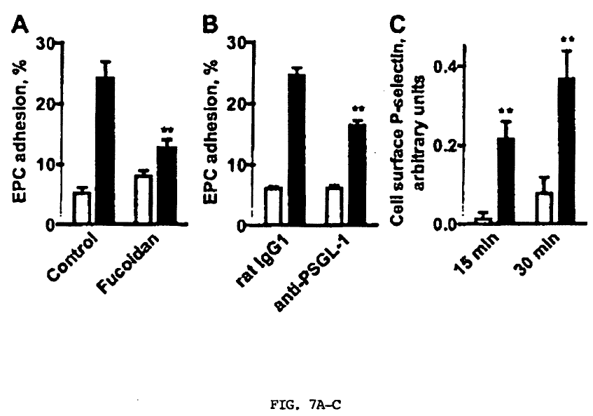

Role of P-Selectin Glycoprotein Ligand-1 and P-Selectin in the Mechanism of Adenosine-Dependent EPC Adhesion to Cardiac Microvascular Endothelium. Because the P-selectin glycoprotein ligand (PSGL)-1 has been previously implicated in eEPC adhesion to the vascular wall (Vajkoczy et al., 2003), the inventors investigated its potential role in adenosine receptor-stimulated eEPC adhesion to MCEC-1 cells. Fucoidan, a polysaccharide known to block PSGL-1 interaction with P-selectin (Handa et al., 1991), inhibited NECA-dependent stimulation of eEPC adhesion (FIG. 7A). Furthermore, NECA-induced eEPC adhesion to MCEC-1 cells was partially blocked if mouse eEPCs (10⁶ cells/mL) were preincubated with 10 μg/mL a blocking monoclonal anti-PSGL-1 antibody (clone 2PH1, Fitzgerald Industries) but was not affected by preincubation with a control isotype-matched antibody (FIG. 7B). These data suggest that interaction between PSGL-1 and P-selectin plays a role in adenosine-induced eEPC adhesion. Therefore, the inventors next tested whether stimulation of adenosine receptors on MCEC-1 cells could acutely increase P-selectin expression on the cell surface. Indeed, the inventors results show that stimulation of MCEC-1 cells with 10 μmol/L NECA for 15 or 30 min significantly increased P-selectin expression on the surface of endothelial cells (FIG. 7C).

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods, and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### VI. REFERENCES

The following references, to the extent that they provide exemplary procedural or other details supplementary to those set forth herein, are specifically incorporated herein by reference.
U.S. Pat. No. 4,166,452
U.S. Pat. No. 4,256,108
U.S. Pat. No. 4,265,874
U.S. Pat. No. 4,968,697
U.S. Pat. No. 5,034,381
U.S. Pat. No. 5,063,233
U.S. Pat. No. 5,604,251
Adair et al., Am. J. Physiol., 256:H240-H246, 1989.
Adolfsson et al., Acta Pathol. Microbiol. Immunol. Scand., 90:273-275, 1982.
Adolfsson et al., J. Physiol., 310:529-532, 1981.
Adolfsson, Acta Physiol. Scand., 128:259-266, 1986b.
Adolfsson, Arzneimittel-Forschung, 36:1768-1769, 1986a.
Ahmed, J. Am. Geriatr. Soc., 51(1):123-126, 2003.
Aicher et al., Circulation, 107:2134-2139, 2003.
Asahara et al., Circulation Res., 85(6):221-228, 1999.
Asahara et al., Science, 275:964-967, 1997.
Baraldi et al., Bioorg. Med. Chem. Lett., 4:2539-2544, 1994.
Baraldi et al., Bioorganic Medic. Chem., 15(7):2514-2527, 2007.
Bartunek et al., Nat. Clin. Pract. Cardiovasc. Med., 4:S100-S105, 2007.
Belardinelli et al., Eur. Heart J, 22:1205-1213, 2001.
Berne et al., Fed. Proc., 42:3136-3142, 1983.
Beukers et al., J. Medic. Chem., 47(15):3707-3709, 2004.
Bouma et al., Am. J. Physiol., 270:C522-C529, 1996.
Braunwald, Heart Disease—a Textbook of Cardiovascular Medicine, 1997.
Bristow, Cardiology, 92:3-6, 1999.
Bruns et al., Mol. Pharmacol., 29:331-346, 1986.
Cairns et al., Chest, 108(4):3805, 1995.
Chlopicki et al., Exp. Clin. Cardiol., 3:59-64, 1998.
Cleato et al., Blood, 107: 2736-2744, 2005.
Conte et al., J Heart Lung Transplant., 17:679-685, 1998.
Cristalli et al., J. Med. Chem., 35:2363-2368, 1992.
Cronstein et al., J. Immunol., 92:2201-2206, 1992.
Cronstein, J. Appl. Physiol., 76:5-13, 1994.
Davies et al., Clin. Biochem., 30:479-490, 1997.
De Feo et al., Ital. Heart J, 4:511-513, 2003.
Desai et al., Mol. Pharmacol., 67:1406-1413, 2005.
DiBianco, Am. J. Med., 115:480-488, 2003.
Dumcius et al., Medicina, 39:815-822, 2003.
Durand et al., Ann. Med., 27:311-317, 1995.
Dusseau and Hutchins, Respir. Physiol., 71:33-44, 1988.
Dusseau et al., Circ. Res., 59:163-170, 1986.
Eichhorn and Bristow, Circulation, 94:2285-2296, 1996.
Feoktistov and Biaggioni, Biochem. Pharmacol., 55:627-633, 1998.
Feoktistov et al., Biochem. Pharmacol., 62:1163-1173, 2001.
Feoktistov et al., Circ. Res., 90:531-538, 2002.
Feoktistov et al., Circ. Res., 92:485-492, 2003.
Fredholm et al., Biochem. Pharmacol., 61:443-448, 2001.
Fredholm et al., Pharmacol. Rev., 53:527-552, 2001.
Goodman and Gilman's The Pharmacological Basis Of Therapeutics, Hardman et al. (Eds.), 10^{th} Ed., 32:853-860; 35:891-893, 2001.
Grant et al., Circ. Res., 85:699-706, 1999.
Grant et al., Invest. Opthalmol. Visual Sci., 42:2068-2073, 2001.
Handa et al., GMP-140 (CD62; PADGEM) Binds to Sialosyl-Lea and Sialosyl-Lex, and Sulfated Glycans Modulate this Binding, Biochem Biophys Res Commun., 181:1223-1230, 1991
Hatzopoulos et al., Development, 125:1457-1468, 1998.
Hristov et al., Trends in Cardiovascular Medicine, 13(5):201-206, 2003.
Hudlicka et al., Can. J. Cardiol. 2:120-123, 1986.
Hutchison et al., J. Pharmacol. Exp. Ther., 251:47-55, 1989.
Ii et al., Circulation, 111:1114-1120, 2005.
Handa et al., Biochem Biophys Res Commun. 181:1223-1230, 1991.
Ji and Jacobson, Drug Design and Discovery, 16:217-226, 1999.
Jin et al., J Clin Invest. 100:2849-2857, 1997.
Jones et al., Physiology and Pathophysiology of Leukocyte Adhesion. New York: Oxford University Press; 1995:148-168.
Klotz et al., Naunyn-Schmiedebergs Arch. Pharmacol., 357:1-9, 1998.
Kreckler et al., J. Pharmacol. Exp. Ther., 317:172-180, 2006.
Kupatt et al., Circulation, 112:1117-1122, 2005.
Kupatt et al., FASEB J, 19:1576-1578, 2005.
Lambertucci et al., Nucleo. Nucleic Acids, 22(5-8):809-812, 2003.
Langer et al., Circ. Res., 98:e2-10, 2006.
Leesar et al., Circulation, 95:2500-2507, 1997.
Leesar et al., J. Am. Coll. Cardiol., 42:437-445, 2003.
Lidington et al., Am. J. Pathol., 282:C67-C74, 2002.
Linden et al., Mol. Pharmacol., 56:705-713, 1999.
Lohse et al., Naunyn-Schmiedebergs Arch. Pharmacol., 336:204-210, 1987.
Lopes et al., Br. J. Pharmacol., 141:1006-1014, 2004.
Maemoto et al., Br. J. Pharmacol., 122:1202-1208, 1997.
Mall et al., Lab. Invest., 57:86-93, 1987.
Manoria and Manoria, J Indian Med. Assoc., 101(5):311-312; 315, 2003.
Martin et al., J. Pharmacol. Exp. Ther., 265:201-206, 1993.
Marzilli et al., Circulation, 101:2154-2159, 2000.
Mattfeldt and Mall, Cardiovasc. Res., 17:229-237, 1983.
Merighi et al., Neoplasia, 7:894-903, 2005.
Montesinos et al., Am. J. Pathol., 164:1887-1892, 2004.
Murthy and Karmen, J. Clin. Labor. Analys., 11:125-128, 1997.
Ongini et al., Naunyn-Schmiedebergs Arch. Pharmacol., 359:7-10, 1999.
PCT Appln. WO 98/33791
PCT Appln. WO/2005/078073
Physicians Desk Reference
Remington's Pharmaceutical Sciences” 15th Edition, pages 1035-1038 and 1570-1580, 1990.
Ryzhov et al., J. Pharmacol. Exp. Ther., 382:565-572, 2007.
Schachinger et al., N. Engl. J. Med., 355:1210-1221, 2006.
Shaw et al., Blood Cells, Molecules, and Diseases, 32(1):168-75, 2004.
Symons et al., Circ. Res., 73:503-513, 1993.
Teleron et al., Transfusion, 45:21-25, 2005.
The Merck Index, Thirteenth Edition
Tornling et al., Acta Pathol. Microbiol. Scand., 86:82, 1978.
Tornling et al., Arzneimittel-Forschung, 30:622-623, 1980a.
Tornling et al., Arzneimittel-Forschung, 30:791-792, 1980b.
Tornling, Acta Pathol. Microbiol. Immunol. Scand., 278:1-63, 1982a.
Tornling, Acta Pathol. Microbiol. Immunol. Scand., 90:269-271, 1982b.
Torry et al., Am. J. Physiol., 262:H980-H986, 1992.
Tranquille and Emeis, Thromb. Haemostasis, 66:479-483, 1991.
Vajkoczy et al., J. Exp. Med., 197:1755-1765, 2003.
Vischer and Wollheim, Blood, 91:118-127, 1998.
Vittori et al., Nucleo. Nucleic Acids, 23(1-2):471-481, 2004.
Wei et al., Cancer Cell, 5:477-488, 2004.
Werner et al., NE J Medicine, 353:999-1007, 2005.
Wothe et al., Am. J. Physiol., 282:R295—R302, 2002.
Young et al., In: Handbook of Applied Therapeutics, 7.1-7.12 and 9.1-9.10, 1989.
Zahler et al., Cardiovasc. Res., 28:1366-1372, 1994.
Zeng et al., Drug Dev. Res., 58:405-411, 2003.
Ziada et al., Cardiovasc. Res., 18:724-732, 1984.

What is claimed is:

1. A method of promoting cell adhesion to vascular endothelium in a subject comprising:
(a) identifying a subject in need of neovascularization;
(b) providing a cell expressing adenosine receptors;
(c) contacting the cell with an adenosine receptor ligand, adenosine precursor or adenosine potentiator; and
(d) administering an adenosine receptor ligand, adenosine precursor or potentiator and the cell to the subject.

2. The method of claim 1, wherein said subject suffers from cardiovascular disease.

3. The method of claim 2, wherein said subject suffers from cardiac ischemia.

4. The method of claim 2, wherein said subject suffers from heart failure.

5. The method of claim 1, wherein said cell is a stem cell.

6. The method of claim 5, wherein said stem cell is an endothelial progenitor cell (EPC).

7. The method of claim 5, wherein said stem cell is enriched from or a component of unfractionated bone marrow preparation.

8. The method of claim 5, wherein said stem cell is autologous to said subject.

9. The method of claim 5, wherein said stem cell is heterologous to said subject.

10. The method of claim 1, wherein step (d) comprises intravenous infusion.

11. The method of claim 1, wherein step (d) comprises antegrade infusion into coronary arteries.

12. The method of claim 1, wherein step (d) comprises retrograde infusion via coronary sinus.

13. The method of claim 1, wherein step (d) comprises intracardiac injection.

14. The method of claim 2, further comprising the step of obtaining said stem cell.

15. The method of claim 14, wherein obtaining said stem cell comprises collection of tissue, bone marrow or peripheral blood and cell fractionation.

16. The method of claim 15, wherein said stem cell is cultured prior to step (c).

17. The method of claim 1, wherein said adenosine receptor ligand is adenosine.

18. The method of claim 1, wherein said adenosine receptor ligand is an adenosine receptor agonist.

19. The method of claim 1, wherein said adenosine ligand is adenosine resulted from breakdown of AMP, ADP or ATP.

20. The method of claim 1, wherein said adenosine ligand is adenosine resulted from inhibition of adenosine reuptake.

21. The method of claim 1, wherein said adenosine ligand is adenosine resulted from modulation of adenosine metabolism.

22. The method of claim 1, wherein said adenosine precursor is AMP, ADP or ATP.

23. The method of claim 1, wherein said adenosine potentiator is an inhibitor of adenosine reuptake or a modulator of adenosine metabolism.

24. A method of promoting cell adhesion to vascular endothelium in a subject comprising:
(a) identifying a subject in need of muscle tissue regeneration;
(b) administering to said subject:
(i) a cell having the ability to regenerate muscle tissue; and
(ii) an adenosine receptor ligand, adenosine precursor or adenosine potentiator.

25. The method of claim 24, wherein said subject suffers from cardiovascular disease.

26. The method of claim 25, wherein said subject suffers from cardiac ischemia.

27. The method of claim 25, wherein said subject suffers from heart failure.

28. The method of claim 24, wherein said cell is a stem cell.

29. The method of claim 28, wherein said stem cell is an endothelial progenitor cell (EPC).

30. The method of claim 28, wherein said stem cell is enriched from or a component of unfractionated bone marrow preparation.

31. The method of claim 28, wherein said stem cell is autologous to said subject.

32. The method of claim 28, wherein said stem cell is heterologous to said subject.

33. The method of claim 24, wherein step (b)(i) comprises intravenous infusion.

34. The method of claim 24, wherein step (b)(i) comprises antegrade infusion into coronary arteries.

35. The method of claim 24, wherein step (b)(i) comprises retrograde infusion via coronary sinus.

36. The method of claim 24, wherein step (b)(i) comprises intracardiac injection.

37. The method of claim 25, further comprising the step of obtaining said stem cell.

38. The method of claim 26, wherein obtaining said stem cell comprises collection of tissue, bone marrow or peripheral blood and cell fractionation.

39. The method of claim 25, wherein said stem cell is cultured prior to step (c).

40. The method of claim 24, wherein said adenosine receptor ligand is adenosine.

41. The method of claim 24, wherein said adenosine receptor ligand is an adenosine receptor agonist.

42. The method of claim 24, wherein said adenosine ligand is adenosine resulted from breakdown of AMP, ADP or ATP.

43. The method of claim 24, wherein said adenosine ligand is adenosine resulted from inhibition of adenosine reuptake.

44. The method of claim 24, wherein said adenosine ligand is adenosine resulted from modulation of adenosine metabolism.

45. The method of claim 24, wherein said adenosine precursor is AMP, ADP or ATP.

46. The method of claim 24, wherein said adenosine potentiator is an inhibitor of adenosine reuptake or a modulator of adenosine metabolism.
